(19) United States
(12) Patent Application Publication
(10) Pub. No.: US 2021/0371868 A1
(43) Pub. Date: Dec. 2, 2021

(54) FLOWERING TIME-REGULATING GENE CMP1 AND RELATED CONSTRUCTS AND APPLICATIONS THEREOF

(71) Applicants: PIONEER OVERSEAS CORPORATION, JOHNSTON, IA (US); SINOBIOWAY BIO-AGRICULTURE GROUP CO. LTD., Beijing (CN)

(72) Inventors: RONGRONG JIAO, BEIJING (CN); GUIHUA LU, BEIJING (CN); GUANFAN MAO, BEIJING (CN); CHANGGUI WANG, BEIJING (CN); GUOKUI WANG, BEIJING (CN); JIANTAO WANG, BEIJING (CN)

(73) Assignees: PIONEER OVERSEAS CORPORATION, JOHNSTON, IA (US); SINOBIOWAY BIO-AGRICULTURE GROUP CO LTD, Beijing (CN)

(21) Appl. No.: 16/957,994

(22) PCT Filed: Dec. 27, 2018
(86) PCT No.: PCT/CN2018/124381

§ 371 Date: Jun. 25, 2020

(30) Foreign Application Priority Data
Dec. 28, 2017 (CN) 201711459345.3

(51) Int. Cl. C12N 15/82; C07K 14/415

(52) U.S. Cl. CPC C12N 15/827; C12N 15/8218; C07K 14/415

(57) ABSTRACT

Isolated polynucleotides and polypeptides, and recombinant DNA constructs and CRISPR-Cas constructs useful for regulating plant heading date or flowering time are provided, and methods utilizing these recombinant DNA constructs and CRISPR-Cas constructs are also provided. Compositions comprising these recombinant DNA constructs or a modified endogenous CMP1 gene are further provided.

### FIELD

This disclosure relates to the field of plant breeding and genetics and, in particular, relates to recombinant DNA constructs and genome editing constructs useful for regulating flowering time and/or heading date of plants, and methods for the control of flowering time and/or heading date in plants.

### BACKGROUND

The growth phase of plants generally includes a vegetative growth phase and a reproductive growth phase. The transition from vegetative to reproductive growth is affected by various flowering signals. The flowering signals are affected by various factors, such as genetic factors (e.g., genotype) and environmental factors (e.g., photoperiod and light intensity) (Dung et al., Theoretical and Applied Genetics, 97: 714-720 (1998)).

Flowering time or heading date is an important agronomic trait and is a critical determinant of the distribution and regional adaptability of plants. Most angiosperm species are induced to flower in response to environmental stimuli (e.g, day length and temperature) and internal cues including age.

From the genetic perspective, two phenotypic changes that control vegetative and floral growth are programmed in the plant. The first genetic change involves the switch from the vegetative to the floral state. If this genetic change is not functioning properly, flowering may not occur. The second genetic event follows the commitment of the plant to form flowers. The observation that the organs of the plant develop on a sequential manner suggests that a genetic mechanism exists in which a series of genes are sequentially turned on and off.

Studies of two distantly related dicotyledons, Arabidopsis thaliana and Antirrhinum majus, has led to the identification of three classes of homeotic genes, acting alone or in combination to determine floral organ identity (Bowman, et al., Development, 112:1 (1991); Carpenter and Coen, Genes Devl., 4:1483 (1990); Schwarz-Sommer, et al., Science, 250: 931 (1990)). Several of these genes are transcription factors whose conserved DNA-binding domain has been designated as MADS box (Schwarz-Sommer, et al., supra).

Earlier acting genes that control the identity of flower meristem have also been characterized. Flower meristems are derived from inflorescence meristem in both Arabidopsis and Antirrhinum. Two factors that control the development of meristematic cells into flowers are known. In Arabidopsis, the factors are the products of the LEAFY gene (Weige, et al. Cell 69:843 (1992)) and the APETALA1 gene (Mandel, et al., Nature 360:273 (1992)). When either of these genes is inactivated by mutation, structures combining the properties of flowers and inflorescence develop (Weigel, et al., supra; Irish and Sussex, Plant Cell, 2:741 (1990)). In Antirrhinum, the homologue of the Arabidopsis LEAFY gene is FLORICAULA (Coen, et al., Cell, 63:1311 (1990)) and that of the APETALA1 gene is SQUAMOSA (Huijser, et al., EMBO J., 11:1239 (1992)). The latter pair contains MADS box domains.

Accelerating or delaying the onset of flowering can be useful to farmers and seed producers. Accordingly, there is a need to develop new compositions and methods for altering the flowering characteristics of the target plant (e.g., cereals, rice and maize, in warmer climatic zones, and wheat, barley, oats and rye in more temperature climates). This disclosure provides such compositions and methods.

### SUMMARY

In one aspect, the present disclosure includes an isolated polynucleotide regulating plant flowering time, comprising: (a) a polynucleotide with a nucleotide sequence of at least 85% identity to SEQ ID NO: 5; (b) a polynucleotide with a nucleotide sequence of at least 85% identity to SEQ ID NO: 6; (c) a polynucleotide encoding a polypeptide with an amino acid sequence of at least 90% identity to SEQ ID NO: 7; or (d) the full complement of the nucleotide sequence of (a), (b) or (c), wherein increasing expression of the polynucleotide in plants prolongs transition from vegetative growth to reproductive growth; reducing the expression and/or function of the polynucleotide in plants promotes transition from vegetative growth to reproductive growth. In certain embodiments, the polynucleotide comprises the nucleotide sequence of SEQ ID NO: 5 or SEQ ID NO: 6. In certain embodiments, the polynucleotide encodes a polypeptide comprising the amino acid sequence of SEQ ID NO: 7.

In another aspect, the present disclosure provides the use of the isolated flowering time-regulating polynucleotide in a plant to regulate the flowering time, wherein the isolated polynucleotide comprises (a) a polynucleotide with a nucleotide sequence of at least 85% identity to SEQ ID NO: 5; (b) a polynucleotide with a nucleotide sequence of at least 85% identity to SEQ ID NO: 6; (c) a polynucleotide encoding a polypeptide with an amino acid sequence of at least 90% identity to SEQ ID NO: 7; or (d) the full complement of the nucleotide sequence of (a), (b) or (c). Certain embodiments provide for the use of the isolated polynucleotide in a plant to promote earlier flowering time by reducing expression of the polynucleotide and/or reducing the function of the encoded polypeptide. Certain embodiments provide for the use of the isolated polynucleotide in a plant to promote delayed flowering time by increasing expression of the polynucleotide.

In another aspect, the present disclosure includes a recombinant DNA construct comprising the isolated flowering time-regulating polynucleotide operably linked to at least one regulatory element, wherein the polynucleotide comprises (a) a polynucleotide with a nucleotide sequence of at least 85% identity to SEQ ID NO: 5 or 6; (b) a polynucleotide encoding a polypeptide with an amino acid sequence of at least 90% identity to SEQ ID NO: 7; or (c) the full complement of the nucleotide sequence of (a) or (b). In certain embodiments, the at least one regulatory element is a promoter functional in a plant. In certain embodiments the at least one regulatory element is heterologous to the polynucleotide.

In another aspect, the present disclosure includes a modified plant, plant cell or seed with altered expression of a polynucleotide encoding a flowering time-regulating polypeptide CMP1, wherein the plant exhibits altered flowering trait when compared to a control plant planted under the same conditions.

In certain embodiments, the plant, plant cell, or seed is modified to have increased expression of the flowering time-regulating gene CMP1, wherein said plant or plant produced from said plant cell or seed has a delayed flowering time when compared to a control plant not having said increased expression. In certain embodiments, the modified plant, plant cell, or seed comprises a recombinant DNA construct comprising a CMP1 polynucleotide operably linked to at least one regulatory element, wherein the CMP1 polynucleotide comprises (a) a polynucleotide with a nucleotide sequence of at least 85% identity to SEQ ID NO: 5 or 6; (b) a polynucleotide encoding a polypeptide with an amino acid sequence of at least 90% identity to SEQ ID NO: 7; or (c) the full complement of the nucleotide sequence of (a) or (b); thereby increasing expression of the polynucleotide in the modified plant, plant cell, or seed. In certain embodiments, the plant comprises a modified regulatory element, wherein the modified regulatory element increases the expression of an endogenous polynucleotide comprising (a) a polynucleotide with a nucleotide sequence of at least 85% identity to SEQ ID NO: 5; (b) a polynucleotide with a nucleotide sequence of at least 85% identity to SEQ ID NO: 6; (c) a polynucleotide encoding a polypeptide with an amino acid sequence of at least 90% identity to SEQ ID NO: 7; or (d) the full complement of the nucleotide sequence of (a), (b) or (c).

In certain embodiments, the plant, plant cell, or seed is modified to have reduced expression and/or reduced function of the flowering time-regulating gene CMP1, wherein said plant, or plant produced from said plant cell or seed has an earlier flowering time when compared to a control plant not having said reduced expression and function. In certain embodiments, the plant comprises a suppression DNA construct, wherein the suppression DNA construct comprises a suppression element operably linked to at least one heterologous regulatory element, wherein the suppression element comprises at least 100 contiguous base pairs of (a) a polynucleotide with a nucleotide sequence of at least 85% identity to SEQ ID NO: 5 or 6; (b) a polynucleotide encoding a polypeptide with an amino acid sequence of at least 90% identity to SEQ ID NO: 7; or (c) the full complement of the nucleotide sequence of (a) or (b).

In certain embodiments, the plant, plant cell, or seed is modified to have decreased function and/or activity of the flowering time-regulating gene CMP1, thereby promoting earlier flowering time when compared to a control plant. In certain embodiments, the plant comprises a modified flowering time-regulating gene CMP1 or its promoter by (a) introducing a DNA fragment or deleting a DNA fragment or replacing a DNA fragment or introducing (b) one or more nucleotide changes in the genomic region comprising the endogenous CMP1 gene and its promoter, wherein the function and/or of an endogenous CMP1 polypeptide is reduced (can be used synonymously with decreased), when compared to the wild-type CMP1 polypeptide from a control plant.

In one embodiment, the plant comprises a mutated CMP1 gene resulting in reduced expression and/or function. In another embodiment, the plant comprises a mutated CMP1 promoter resulting in reduced expression and/or function.

In certain embodiments, the plant for the use in the compositions and methods provided herein is selected from the group consisting of rice, maize, soybean, sunflower, sorghum, canola, wheat, alfalfa, cotton, barley, millet, sugar cane and switchgrass.

In another aspect, a rice plant is provided, wherein the rice plant comprises a modified genomic locus, wherein expression of an endogenous polynucleotide encoding the polypeptide having an amino acid sequence of at least 90% sequence identity compared to SEQ ID NO: 7 is increased, or the function of an endogenous polynucleotide encoding the polypeptide having an amino acid sequence of at least 90% identity to SEQ ID NO: 7 is decreased, wherein the time required to flower is reduced when the function of the endogenous polynucleotide is reduced and the time required to flower is increased when the expression of the endogenous polynucleotide is increased. In certain embodiments, the modified genomic locus comprises a mutation in a regulatory region that reduces the expression of the endogenous polynucleotide. In certain embodiments, the modified genomic locus comprises a mutation in a gene that decreases the function and/or activity of the endogenous polynucleotide.

In another aspect, methods are provided for regulating plant flowering time, comprising altering the expression or function of a polynucleotide encoding a CMP1 polypeptide in a plant (e.g., rice), wherein the polynucleotide comprises: (a) a polynucleotide with a nucleotide sequence of at least 85% identity to SEQ ID NO: 5; (b) a polynucleotide with a nucleotide sequence of at least 85% identity to SEQ ID NO: 6; and (c) a polynucleotide encoding a polypeptide with an amino acid sequence of at least 90% identity to SEQ ID NO: 7. In certain embodiments, the method comprises increasing expression of the polynucleotide in the said plants thereby prolonging transition from vegetative growth to reproductive growth. In certain embodiments, the method comprises reducing expression of the polynucleotide or decrease the function and/or activity of the polynucleotide in the said plants thereby promoting transition from vegetative growth to reproductive growth.

In certain embodiments, the increase in expression or the decrease in function and/or activity of the polynucleotide is altered by a step selected from the group consisting of: (a) increasing expression of a polynucleotide encoding a CMP1 polypeptide in plant by using a recombinant DNA construct comprising a polynucleotide encoding the CMP1 polypeptide operably linked to at least one heterologous regulatory element, wherein the polynucleotide encodes a polypeptide having an amino acid sequence of at least 90% identity compared to SEQ ID NO: 7; (b) increasing the expression of an endogenous polynucleotide encoding the polypeptide having an amino acid sequence of at least 90% sequence identity compared to SEQ ID NO: 7 using a CRISPR-Cas construct; (c) reducing the function and/or activity of an endogenous polynucleotide encoding the polypeptide having an amino acid sequence of at least 90% sequence identity compared to SEQ ID NO: 7; and (d) decreasing the expression of a polynucleotide encoding a CMP1 polypeptide in a plant by using a suppression DNA construct, wherein the suppression DNA construct comprises a suppression element that reduces the expression of an endogenous polynucleotide encoding the polypeptide having an amino acid sequence of at least 90% identity compared to SEQ ID NO: 7.

In another aspect, methods are provided for making a plant in which the expression or the activity of an endogenous CMP1 polypeptide is increased, as compared to the activity of the wild-type CMP1 polypeptide from a control plant. In certain embodiments, the method comprises the step of (i) introducing a DNA fragment which can increase the expression of CMP1 or (ii) introducing one or more nucleotide changes in the genomic region comprising the endogenous CMP1 gene, wherein the change is effective for increasing the expression or the activity of the endogenous CMP1 polypeptide.

In another aspect, provided are method for making a plant in which the function and/or activity of an endogenous CMP1 polypeptide is reduced when compared to the wild-type CMP1 polypeptide from a control plant. In certain embodiments, the method comprises the step of (i) introducing a DNA fragment, deleting a DNA fragment or replacing a DNA fragment, or (ii) introducing one or more nucleotide changes in the genomic region comprising the endogenous CMP1 gene and its promoter, wherein the modification is effective for making the function of the endogenous CMP1 polypeptide lost.

In certain embodiments of the methods described herein, the change (e.g., increased or reduced) in expression, function and/or activity of the endogenous CMP1 polypeptide is introduced using zinc finger nuclease, Transcription Activator-Like Effector Nuclease (TALEN), CRISPR-Cas, guided Cas endonuclease, meganuclease or CRISPR-Cas ribonucleoprotein complexes.

In another aspect, a method of identifying one or more alleles associated with late flowering time in a population of plants (e.g., rice) is provided, wherein the method comprises the steps of: (a) detecting in a population of plants one or more polymorphisms in (i) a genomic region encoding a polypeptide or (ii) a regulatory region controlling expression of the polypeptide, wherein the polypeptide comprises the amino acid sequence selected from the group consisting of SEQ ID NO: 7 or a sequence that is 90% identical to SEQ ID NO: 7, wherein the one or more polymorphisms in the genomic region encoding the polypeptide or in the regulatory region controlling expression of the polypeptide is associated with late flowering time; and (b) identifying one or more alleles at the one or more polymorphisms that are associated with late flowering time. Wherein the one or more alleles associated with late flowering time is used for marker assisted selection of a plant with late flowering time.

### BRIEF DESCRIPTION OF THE DRAWINGS AND SEQUENCE LISTINGS

The disclosure can be more fully understood from the following detailed description and the accompanying drawings and Sequence Listing which form a part of this application.

FIG. 1 shows the relative expression levels of OsCMP1 gene in leaves of different transgenic rice lines (DP2300) by real-time PCR analyses. The base expression level in ZH11-TC is set at 1.00, the numbers on the top of the columns are fold-change compared to ZH11-TC rice.

FIG. 2 shows the schematic of sgRNA distribution in the genome of a rice OsCMP1 gene.

FIG. 3 shows an example of single sgRNA distribution in the genome of a rice OsCMP1 gene.

FIG. 4 shows an example of two sgRNAs distribution in the genome of a rice OsCMP1 gene.

FIG. 5 shows an alignment of mutations induced by the CRISPR-Cas construct DP2855 in rice plants. The mutations were identified by PCR and sequencing. The reference sequence represents the unmodified locus with each target site underlined. The PAM sequence and expected site of cleavage are also indicated. Deletion, insertion or replacement is shown by a “-”, an italicized underlined nucleotide or bolded italicized nucleotide, respectively.

FIG. 6 shows an alignment of mutations induced by CRISPR-Cas construct DP2925. The mutations were identified by PCR and sequencing. The reference sequence represents the unmodified locus with each target site underlined. The PAM sequence and expected site of cleavage are also indicated. Deletion, insertion or replacement is shown by a “-”, an italicized underlined nucleotide or bolded italicized nucleotide, respectively.

FIG. 7 shows the schematic of gCAS-gRNA-DsRed segregation process in progeny.

TABLE 1
SEQ ID NOs for nucleotide and amino acid sequences
provided in the sequence listing

| | | SEQ ID NO: | SEQ ID NO: |
|---|---|---|---|
| Source species | Clone Description | (Nucleotide) | (Amino Acid) |
| Oryza sativa | T-DNA flanking | 1 | n/a |
| | sequence in | | |
| | AH41120 (RB) | | |
| Oryza sativa | T-DNA flanking | 2 | n/a |
| | sequence in | | |
| | AH41120 (LB) | | |
| Oryza sativa | T-DNA flanking | 3 | n/a |
| | sequence in | | |
| | AH11081 (RB) | | |
| Oryza sativa | T-DNA flanking | 4 | n/a |
| | sequence in | | |
| | AH11081 (LB) | | |
| Oryza sativa | OsCMP1 | 5, 6 | 7 |
| Oryza sativa | OsCMP1 regulatory | 8 | n/a |
| | element | | |
| Artificial | Primers | 9-12 | n/a |
| Zea mays | Ubiquitin Promoter | 13 | n/a |
| Artificial | Nucleus localization | 14 | n/a |
| | sequence | | |
| Cauliflower mosaic | CaMV 3′UTR | 15 | n/a |
| Oryza sativa | rU6-Promoter | 16 | n/a |
| Artificial | gRNA scaffold | 17 | n/a |
| Artificial | gRNA | 18-39 | n/a |
| Oryza sativa | Mutation sequence | 40-69 | n/a |

The sequence descriptions and Sequence Listing attached hereto comply with the rules governing nucleotide and/or amino acid sequence disclosures in patent applications as set forth in 37 C.F.R. § 1.821-1.825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (2):345-373 (1984) which are herein incorporated by reference. The symbols and format used for nucleotide and amino acid sequence data comply with the rules set forth in 37 C.F.R. § 1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

As used herein:

The term “OsCMP1” is a CCT motif family protein 1 (CMP1) and refers to a rice polypeptide that regulates rice flowering trait and is encoded by the rice gene locus LOC_Os07g15770.1. “CMP1 polypeptide” refers herein to the OsCMP1 polypeptide and its homologs from other organisms.

The OsCMP1 polypeptide (SEQ ID NO: 7) is encoded by the coding sequence (CDS) (SEQ ID NO: 6) or nucleotide sequence (SEQ ID NO: 5) at rice gene locus LOC_Os07g15770.1. This polypeptide is annotated as “CCT motif family protein, expressed” in TIGR (the internet at plant biology msu.edu/index.shtml), however does not have any prior assigned function.

The terms “monocot” and “monocotyledonous plant” are used interchangeably herein. A monocot of the current disclosure includes the gramineae.

The terms “dicot” and “dicotyledonous plant” are used interchangeably herein. A dicot of the current disclosure includes the following families: Brassicaceae, Leguminosae, and Solanaceae.

“Flowering” refers to the process of anthesis, i.e. glume dehiscent and anthers scattering under suitable temperature and humidity, or the process of flower formation. Herein flowering is used to referring the process from young panicle differentiation, maturation, to the panicle heading.

“Flower development” or “floral development” is intended to mean the development of a flower or inflorescence from the initiation of the floral meristem to the development of the mature flower.

“Reproductive development” is intended to mean the development of a flower or inflorescence from the initiation of the floral meristem through pollination and the development of mature fruit.

Plants having an “early flowering time” as used herein are plants which start to flower earlier than control plants. Hence this term refers to plants that show an earlier start of flowering. Flowering time of plants can be assessed by counting the number of days (time to flower) between sowing and the emergence of a first inflorescence. The “flowering time” of a plant can be readily determined using known methods and standards.

“Heading” as used herein refers to the process of cereal panicle extended from flag leaf sheath.

“Heading date” and “heading time” are used interchangeably herein and refers to the number of days from the day of seeding to the day when the main stem panicle of an individual plant extended from the flag leaf sheath, or 50% young panicles of plants in one row head out the flag leaf sheath. Heading date is an important agronomic trait, which is under the regulation of basic nutritional genes and photoperiod-sensitivity genes and plays a key role in the adaptation and geographic distribution of rice varieties. Appropriate heading date is a prerequisite for attaining the desired yield level.

The rice panicle will flower after the panicle headed out under normal conditions. Herein heading date will be used to indicate the flowering time.

The maturity date is the date when 90% glume, grain spikelet axis or vice glume become yellow from appearance, which is the best harvest period.

“Plant height” as used herein refers to the height from the surface of the field to the top of the highest panicle or leaf of an individual plant.

The terms “full complement” and “full-length complement” are used interchangeably herein and refer to a complement of a given nucleotide sequence, wherein the complement and the nucleotide sequence consist of the same number of nucleotides and are 100% complementary.

The term “trait” refers to a physiological, morphological, biochemical, or physical characteristic of a plant or particular plant material or cell.

“Agronomic characteristic” is a measurable parameter including but not limited to: greenness, grain yield, growth rate, total biomass or rate of accumulation, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, panicle size, early seedling vigor and seedling emergence under low temperature stress.

“Transgenic” refers to any cell, cell line, callus, tissue, plant part or plant, the genome of which has been altered by the presence of a recombinant DNA construct.

A “control” or “control plant” or “control plant cell” provides a reference point for measuring changes in phenotype of a subject plant or plant cell which was genetically altered by, such as transformation, and has been affected as to a gene of interest. A subject plant or plant cell may be descended from a plant or cell so altered and will comprise the alteration.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed sergeant among progeny of a subject plant or plant cell; (d) a plant or plant cell genetically identical to the subject plant or plant cell but which is not exposed to a condition or stimulus that would induce expression of the gene of interest; or (e) the subject plant or plant cell itself, under conditions in which the gene of interest is not expressed.

In this disclosure, ZH11-WT, ZH11-TC, WT and empty vector plants may be designated as control plants. ZH11-WT represents wild type Zhonghua 11, ZH11-TC represents rice plants generated from tissue cultured Zhonghua 11, WT represents the wild type plants, such as Zhonghua 11, Daohuaxiang 2, and empty vector represents plants transformed with empty vector DP0158.

“Genome” as it applies to plant cells encompasses not only chromosomal DNA found within the nucleus, but organelle DNA found within subcellular components (e.g., mitochondrial, plastid) of the cell.

An “allele” is one of two or more alternative forms of a gene occupying a given locus on a chromosome. When the alleles present at a given locus on a pair of homologous chromosomes in a diploid plant are the same, that plant is homozygous at that locus. If the alleles present at a given locus on a pair of homologous chromosomes in a diploid plant differ, that plant is heterozygous at that locus. If a transgene is present on one of a pair of homologous chromosomes in a diploid plant, that plant is hemizygous at that locus.

The term “gene” refers to a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. A mutated or modified plant is a plant comprising a mutated gene.

As “targeted mutation” is a mutation in a native gene that was made by altering a target sequence within the native gene using a method involving a double-strand-break-inducing agent that is capable of inducing a double-strand break in the DNA of the target sequence as disclosed herein of known in the art.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Progeny” comprises any subsequent generation of a plant.

“Heterologous” with respect to sequence means a sequence that originates from a foreign species, or, if from the same species, is substantially modified from its native form in composition and/or genomic position by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases.

The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified but does not encompass the alteration of the cell or vector by naturally occurring events (e.g., spontaneous mutation, natural transformation/transduction/transposition) such as those occurring without deliberate human intervention.

“Non-genomic nucleic acid sequence” or “non-genomic nucleic acid molecule” or “non-genomic polynucleotide” refers to a nucleic acid molecule that has one or more change in the nucleic acid sequence compared to a native or genomic nucleic acid sequence. In some embodiments the change to a native or genomic nucleic acid molecule includes but is not limited to: changes in the nucleic acid sequence due to the degeneracy of the genetic code; codon optimization of the nucleic acid sequence for expression in plants; changes in the nucleic acid sequence to introduce at least one amino acid substitution, insertion, deletion and/or addition compared to the native or genomic sequence; removal of one or more intron associated with the genomic nucleic acid sequence; insertion of one or more heterologous introns; deletion of one or more upstream or downstream regulatory regions associated with the genomic nucleic acid sequence; insertion of one or more heterologous upstream or downstream regulatory regions; deletion of the 5′ and/or 3′ untranslated region associated with the genomic nucleic acid sequence; insertion of a heterologous 5′ and/or 3′ untranslated region; and modification of a polyadenylation site. In some embodiments, the non-genomic nucleic acid molecule is a cDNA. In some embodiments, the non-genomic nucleic acid molecule is a synthetic nucleic acid sequence.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source but arranged in a manner different than that normally found in nature.

“Regulatory sequences” and “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably and refer to a promoter that is expressed predominantly but not necessarily exclusively in one tissue or organ, but that may also be expressed in one specific cell.

“Developmentally regulated promoter” refers to a promoter whose activity is determined by developmental events.

“Genetic modification” refers to a change or alteration in the genomic nucleic acid sequence of a plant introduced by deliberate human activity.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Transient transformation” refers to the introduction of a nucleic acid fragment into the nucleus, or DNA-containing organelle, of a host organism resulting in gene expression without genetically stable inheritance.

“Stable transformation” refers to the introduction of a nucleic acid fragment into a genome of a host organism resulting in genetically stable inheritance.

A “nuclear localization signal” is a signal peptide which directs the protein to the nucleus (Raikhel. (1992) Plant Phys. 100:1627-1632).

A “suppression DNA construct” is a recombinant DNA construct which when transformed or stably integrated into the genome of the plant, results in “silencing” of a target gene in the plant. The target gene may be endogenous or transgenic to the plant. “Silencing”, as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, includes lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

“Antisense inhibition” refers to the production of antisense RNA transcripts capable of suppressing the expression of the target gene or gene product.

“Cosuppression” refers to the production of sense RNA transcripts capable of suppressing the expression of the target gene or gene product. “Sense” RNA refers to RNA transcript that includes the mRNA and can be translated into protein within a cell or in vitro.

Another variation describes the use of plant viral sequences to direct the suppression of proximal mRNA encoding sequences (PCT Publication No. WO 98/36083 published on Aug. 20, 1998).

RNA interference (RNAi) refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants.

MicroRNAs (miRNAs) are designed that regulate target genes (e.g., the polynucleotide sequences disclosed herein) by binding to complementary sequences located in the transcripts produced by these genes for example by translational inhibition and RNA cleavage.

“CRISPR-associated genes” refers to nucleic acid sequences that encode polypeptide components of clustered regularly interspersed short palindromic repeats (CRISPR)-associated systems (Cas), and the genes are generally coupled, associated or close to or in the vicinity of flanking CRISPR loci. The terms “Cas gene”, “CRISPR-associated gene” are used interchangeably herein. Examples include, but are not limited to, Cas3 and Cas9, which encode endonucleases from the CRISPR type I and type II systems, respectively.

“Cas endonuclease” refers to a Cas protein encoded by a Cas gene, wherein said Cas protein is capable of introducing a double strand break into a DNA target sequence. The Cas endonuclease is guided by the guide polynucleotide to recognize and optionally introduce a double strand break at a specific target site into the genome of a cell.

“Guide RNA (gRNA)” refers to a crRNA (CRISPR RNA):tracrRNA fused hybrid RNA molecule encoded by a customizable DNA element that, generally, comprises a copy of a spacer sequence which is complementary to the protospacer sequence of the genomic target site, and a binding domain for an associated-Cas endonuclease of the CRISPR complex.

“Guide polynucleotide” refers to a polynucleotide sequence that can form a complex with a Cas endonuclease and enables the Cas endonuclease to recognize and optionally cleave a DNA target site. The guide polynucleotide can be comprised of a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA”.

The term “guide polynucleotide/Cas endonuclease system” refers to a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA, but only if the correct protospacer-adjacent motif (PAM) is approximately oriented at the 3′ end of the target sequence.

“Genomic target site” refers to a protospacer and a protospacer adjacent motif (PAM) located in a host genome selected for targeted mutation and/or double-strand break.

“Protospacer” refers to a short DNA sequence (12 to 40 bp) that can be targeted for mutation, and/or double-strand break, mediated by enzymatic cleavage with a CRISPR system endonuclease guided by complementary base-pairing with the spacer sequence in the crRNA or sg RNA.

“Protospacer adjacent motif (PAM)” includes a 3 to 8 bp sequence immediately adjacent to the protospacer sequence in the genomic target site.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop sequence.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 bp by depending on the CRISPR locus (WO2007/025097 published Mar. 1, 2007).

Endonucleases are enzymes that cleave the phosphodiester bond within a polynucleotide chain and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (patent application WO-PCT PCT/US12/30061 filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates.

TAL effector nucleases are a new class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, Foki. The unique, modular TAL effector DNA binding domain allows for the design of proteins with potentially any given DNA recognition specificity (Miller et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs consist of an engineered DNA-binding zinc finger domain linked to a non-specific endonuclease domain, for example nuclease domain from a Type I is endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18 nucleotide recognition sequence.

The terms “target site”, “target sequence”, “target DNA”, “target locus”, “genomic target site”, “genomic target sequence”, and “genomic target locus” are used interchangeably herein and refer to a polynucleotide sequence in the genome (including chloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeable herein to refer to a target sequence that is endogenous or native to the genome of a plant and is at the endogenous or native position of that target sequence in the genome of the plant.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Turning now to the embodiments:

Embodiments include isolated polynucleotides and polypeptides, recombinant DNA constructs (including suppression constructs) useful for regulating plant flowering time, compositions (such as plants or seeds) comprising these recombinant DNA constructs, and methods utilizing these recombinant DNA constructs, CRISPR-Cas constructs useful for regulating flowering time, compositions comprising mutant flowering time-regulating gene and its promoter, and methods utilizing the CRISPR-Cas constructs.

Isolated Polynucleotides and Polypeptides

The present disclosure includes the following isolated polynucleotides and polypeptides:

In some embodiments, polynucleotides are provided encoding CMP1 polypeptides.

In some embodiments, isolated polynucleotides are provided comprising: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, when compared to SEQ ID NO: 7; or (ii) a full complement of the nucleic acid sequence of (i), wherein the full complement and the nucleic acid sequence of (i) consist of the same number of nucleotides and are 100% complementary. In certain embodiments, increasing expression of this polynucleotide prolongs transition from vegetative growth to reproductive growth. In other embodiments, reducing expression of the polynucleotide promotes transition from vegetative growth to reproductive growth. Any of the foregoing isolated polynucleotides may be utilized in any recombinant DNA constructs of the present disclosure.

In some embodiments, isolated polypeptides are provided having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, when compared to SEQ ID NO: 7.

In some embodiments, isolated polynucleotides are provided comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, when compared to SEQ ID NO: 5 or 6; or (ii) a full complement of the nucleic acid sequence of (i). Any of the foregoing isolated polynucleotides may be utilized in any recombinant DNA constructs of the present disclosure. The isolated polynucleotide preferably encodes a flowering time-regulating protein. In certain embodiments, increasing expression of this polynucleotide prolongs transition from vegetative growth to reproductive growth. In other embodiments, reducing expression of the polynucleotide promotes transition from vegetative growth to reproductive growth.

Recombinant DNA Constructs and Suppression DNA Constructs In one aspect, the present disclosure includes recombinant DNA constructs and suppression DNA constructs.

In one embodiment, the recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, when compared to SEQ ID NO: 7; or (ii) a full complement of the nucleic acid sequence of (i).

In another embodiment, the recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity as compared to SEQ ID NO: 5 or 6; or (ii) a full complement of the nucleic acid sequence of (i).

In another embodiment, the recombinant DNA construct comprises a polynucleotide operably linked to at least one regulatory sequence (e.g., a promoter functional in a plant), wherein said polynucleotide encodes a CMP1 protein. This polypeptide regulates flowering time, and may be from, for example, Oryza sativa, Oryza australiensis, Oryza barthii, Oryza glaberrima (African rice), Oryza latifolia, Oryza longistaminata, Oryza meridionalis, Oryza officinalis, Oryza punctata, Oryza rufipogon (brownbeard or red rice), Oryza nivara (Indian wild rice), Arabidopsis thaliana, Zea mays, Glycine max, Glycine tabacina, Glycine soja or Glycine tomentella.

In another aspect, the present disclosure includes suppression DNA constructs.

In certain embodiments, the suppression DNA construct comprises at least one regulatory element (e.g., a promoter functional in a plant) operably linked to one or more suppression elements, wherein the suppression element comprises at least 100 contiguous base pairs of (a) (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 7, or (ii) a full complement of the nucleic acid sequence of (a)(i); or (b) a region derived from a sense strand or antisense strand of a target gene of interest, wherein said target gene of interest encodes a flowering time-regulating polypeptide CMP1; or (c) (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 5, or (ii) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 6, or (iii) a full complement of the nucleic acid sequence of (c)(i) or (c)(ii).

In certain embodiments the suppression constructs comprise RNAi constructs (e.g., siRNA, miRNA optionally operably linked to a regulatory element.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

CRISPR-Cas Constructs:

Provided is a CRISPR-Cas construct comprising: a polynucleotide encoding a CRISPR enzyme, a polynucleotide encoding nuclear localization signal and at least one regulatory sequence operably linked to gRNA, wherein the gRNA is targeted to the genomic region containing endogenous CMP1 gene and its promoter.

In certain embodiments, the gRNA is targeted to the genomic region containing a polynucleotide comprising a nucleotide sequence of at least 95% identity to SEQ ID NO: 5, or 6.

The sgRNA sequences may be distributed anywhere on the target genomic sequences including the promoter, exon, intron, 5′-UTR, and 3′-UTR. In certain embodiments, the sgRNA sequence is selected from the group consisting of SEQ ID NOs:18-39.

In certain embodiments, one sgRNA is used to make the CRISPR-Cas construct. The single sgRNA can guide the Cas9 enzyme to the target region and generate the double strand break at the target DNA sequence, non-homologous end-joining (NHEJ) repairing mechanism and homology directed repair (HDR) will be triggered, and it often induces random insertion, deletion and substitution at the target site.

In certain embodiments, two sgRNAs can be used to make the CRISPR-Cas construct. This construct can lead to fragment deletion, point mutation (small insertion, deletion and substitution).

Regulatory Elements:

A recombinant DNA construct (including a suppression DNA construct) of the present disclosure may comprise at least one regulatory element.

A regulatory element may be a promoter, enhancer, 5′UTR, or 3′UTR.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may (or may not) have pleiotropic effects, although candidate gene efficacy may be estimated when driven by a constitutive promoter. Use of tissue-specific and/or stress-specific promoters may eliminate undesirable effects, but retain the ability to regulate plant flowering time. This type of effect has been observed in Arabidopsis for drought and cold tolerance (Kasuga et al., Nature Biotechnol. 17:287-91 (1999)).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter which causes the desired temporal and spatial expression may be used in the methods of the present disclosure.

For the expression of a polynucleotide in developing seed tissue, promoters of particular interest include seed-preferred promoters, particularly early kernel/embryo promoters and late kernel/embryo promoters. Kernel development post-pollination is divided into approximately three primary phases. The lag phase of kernel growth occurs from about 0 to 10-12 DAP. During this phase the kernel is not growing significantly in mass, but rather important events are being carried out that will determine kernel vitality (e.g., number of cells established). The linear grain fill stage begins at about 10-12 DAP and continues to about 40 DAP. During this stage of kernel development, the kernel attains almost all of its final mass, and various storage products (i.e., starch, protein, oil) are produced. Finally, the maturation phase occurs from about 40 DAP to harvest. During this phase of kernel development, the kernel becomes quiescent and begins to dry down in preparation for a long period of dormancy prior to germination. As defined herein “early kernel/embryo promoters” are promoters that drive expression principally in developing seed during the lag phase of development (i.e., from about 0 to about 12 DAP). “Late kernel/embryo promoters”, as defined herein, drive expression principally in developing seed from about 12 DAP through maturation. There may be some overlap in the window of expression. The choice of the promoter will depend on the ABA-associated sequence utilized and the phenotype desired.

Early kernel/embryo promoters include, for example, Cim1 that is active 5 DAP in particular tissues (WO 00/11177), which is herein incorporated by reference. Other early kernel/embryo promoters include the seed-preferred promoters end1 which is active 7-10 DAP, and end2, which is active 9-14 DAP in the whole kernel and active 10 DAP in the endosperm and pericarp (WO 00/12733), herein incorporated by reference. Additional early kernel/embryo promoters that find use in certain methods of the present disclosure include the seed-preferred promoter Itp2 (U.S. Pat. No. 5,525,716); maize Zm40 promoter (U.S. Pat. No. 6,403,862); maize nuc1c (U.S. Pat. No. 6,407,315); maize ckx1-2 promoter (U.S. Pat. No. 6,921,815 and US Patent Application Publication Number 2006/0037103); maize lec1 promoter (U.S. Pat. No. 7,122,658); maize ESR promoter (U.S. Pat. No. 7,276,596); maize ZAP promoter (U.S. Patent Application Publication Numbers 20040025206 and 20070136891); maize promoter eep1 (U.S. Patent Application Publication Number 20070169226); and maize promoter ADF4 (U.S. Patent Application No. 60/963,878, filed 7 Aug. 2007). Additional promoters for regulating the expression of the nucleotide sequences of the present disclosure in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters for use in the current disclosure may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (GenBank Accession No. EF030817), and the constitutive promoter GOS2 from Zea mays. Other promoters include root preferred promoters, such as the maize NAS2 promoter, the maize Cyclo promoter (US Publication No. 2006/0156439, published Jul. 13, 2006), the maize ROOTMET2 promoter (WO 2005/063998, published Jul. 14, 2005), the CR1 BIO promoter (WO 2006/055487, published May 26, 2006), the CRWAQ81 promoter (WO 2005/035770, published Apr. 21, 2005) and the maize ZRP2.47 promoter (NCBI Accession No. U38790; NCBI GI No. 1063664).

Recombinant DNA constructs of the present disclosure may also include other regulatory elements including, but not limited to, translation leader sequences, introns, and polyadenylation recognition sequences. In another embodiment of the present disclosure, a recombinant DNA construct of the present disclosure further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, Mol. Cell Biol. 8:4395-4405 (1988); Callis et al., Genes Dev. 1:1183-1200 (1987)).

An enhancer or enhancer element refers to a cis-acting transcriptional regulatory element, a.k.a. cis-element, which confers an aspect of the overall expression pattern, but is usually insufficient alone to drive transcription, of an operably linked polynucleotide sequence. An isolated enhancer element may be fused to a promoter to produce a chimeric promoter cis-element, which confers an aspect of the overall modulation of gene expression. Enhancers are known in the art and include the SV40 enhancer region, the CaMV 35S enhancer element, and the like. Some enhancers are also known to alter normal regulatory element expression patterns, for example, by causing a regulatory element to be expressed constitutively when without the enhancer, the same regulatory element is expressed only in one specific tissue or a few specific tissues. Duplicating the upstream region of the CaMV35S promoter has been shown to increase expression by approximately tenfold (Kay, R. et al., (1987) Science 236: 1299-1302).

Compositions:

Provided are plants comprising in their genome any of the recombinant DNA constructs (including any of the suppression DNA constructs) of the present disclosure (such as any of the constructs discussed above). In certain embodiments, the recombinant DNA constructs comprise heterologous regulatory elements. Compositions also include any progeny of the plant, and any seed obtained from the plant or its progeny, wherein the progeny or seed comprises within its genome the recombinant DNA construct (or suppression DNA construct). Progeny includes subsequent generations obtained by self-pollination or out-crossing of a plant. Progeny also includes hybrids and inbreds.

Also provided are plants in which the expression or the activity of an endogenous CMP1 polypeptide is decreased, when compared to the expression or the activity of wild-type CMP1 polypeptide from a control plant, wherein the plant exhibits early flowering time compared to the control plant. In certain embodiments, the expression or the activity of an endogenous CMP1 polypeptide is decreased through an introduced genetic modification comprising (a) introducing a DNA fragment or deleting a DNA fragment or replacing a DNA fragment or (b) introducing one or more nucleotide changes in the genomic region comprising the endogenous CMP1 gene and its promoter, wherein the change is effective for decreasing the expression or the activity of the endogenous CMP1 polypeptide.

Further provided are plants comprising a modified endogenous CMP1 gene, or a plant in which the endogenous CMP1 gene promoter is modified. Compositions also include any progeny of the plant, and any seed obtained from the plant or its progeny, wherein the progeny or seed comprises within its genome the modified CMP1 gene or promoter. Progeny includes subsequent generations obtained by self-pollination or out-crossing of a plant. Progeny also includes hybrids and inbreds.

In hybrid seed propagated crops, mature transgenic plants or genome edited plants can be self-pollinated to produce a homozygous inbred plant. The inbred plant produces seed containing the newly introduced recombinant DNA construct or genome edited sequence. These seeds can be grown to produce plants that would exhibit an altered agronomic characteristic or used in a breeding program to produce hybrid seed, which can be grown to produce plants that would exhibit such an altered agronomic characteristic. The seeds may be maize seeds, or rice seeds.

The plant of the compositions described herein may be a monocotyledonous or dicotyledonous plant, for example, a maize or soybean plant, such as a maize hybrid plant or a maize inbred plant. The plant may also be sunflower, sorghum, canola, wheat, alfalfa, cotton, rice, barley or millet.

The recombinant DNA construct or the suppression DNA construct may be stably integrated into the genome of the plant. The CRISPR-Cas construct may be stably integrated into the genome of the plant. The modification in the gene or promoter may be stably inherited in the plant.

Embodiments include but are not limited to the following:

1. A transgenic plant (for example, a rice, maize or soybean plant) comprising in its genome a recombinant DNA construct comprising a polynucleotide operably linked to at least one heterologous regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, when compared to SEQ ID NO: 7; and wherein said plant exhibits prolonged transition from vegetative growth to reproduction growth (i.e., delayed flowering).

2. A genome edited plant (for example, a rice, maize or soybean plant) comprising a targeted genetic modification that increases the expression of a polynucleotide encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity, when compared to SEQ ID NO: 7; and wherein said plant exhibits prolonged transition from vegetative growth to reproduction growth (i.e., delayed flowering) and wherein the genetic modification modifies a regulatory element or inserts an expression modulating element.

3. A transgenic plant (for example, a rice, maize or soybean plant) comprising in its genome a suppression DNA construct comprising at least one regulatory element operably linked to suppression elements, wherein the suppression elements derives from a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to a sense strand or antisense strand from which said suppression element is derived, and wherein said target gene of interest encodes a CMP1 polypeptide, and wherein said plant exhibits early flowering time when compared to a control plant.

4. A transgenic plant (for example, a rice, maize or soybean plant) comprising in its genome a suppression DNA construct comprising at least one heterologous regulatory element operably linked to at least 100 contiguous base pairs of (a) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 7, or (b) a full complement of the nucleic acid sequence of (a), and wherein said plant exhibits early flowering time when compared to a control plant.

5. A genome edited plant (for example, a rice, maize or soybean plant) comprising in its genome a genetic modification that reduces the expression and/or function of a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 7, wherein said plant exhibits early flowering time when compared to a control plant.

6. The plant of embodiment 1 to 5, wherein the polynucleotide encodes a CMP1 polypeptide, for example from Oryza sativa, Oryza australiensis, Oryza barthii, Oryza glaberrima (African rice), Oryza latifolia, Oryza longistaminata, Oryza meridionalis, Oryza officinalis, Oryza punctata, Oryza rufipogon (brownbeard or red rice), Oryza nivara (Indian wild rice), Arabidopsis thaliana, Cicer arietinum, Solanum tuberosum, Brassica oleracea, Zea mays, Glycine max, Glycine tabacina, Glycine soja or Glycine tomentella.

7. Any progeny of the above plants in embodiments 1 to 6, any seeds of the above plants in embodiments 1 to 6, any seeds of progeny of the above plants in embodiments 1 to 6, and cells from any of the above plants in embodiments 1 to 6 and progeny thereof.

In any of the foregoing embodiments 1 to 7 or any other embodiments of the present disclosure, the recombinant DNA construct may comprise at least one heterologous promoter functional in a plant as a regulatory element.

The examples below describe some representative protocols and techniques for regulating plant flowering time and observing and/or evaluating plants agricultural characteristics under such conditions.

1. Progeny of a transformed plant which is hemizygous with respect to a recombinant DNA construct, such that the progeny is segregating into plants either comprising or not comprising the recombinant DNA construct: the progeny comprising the recombinant DNA construct would be typically measured relative to the progeny not comprising the recombinant DNA construct (i.e., the progeny not comprising the recombinant DNA construct is the control or reference plant).

2. Introgression of a recombinant DNA construct into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a recombinant DNA construct: the second hybrid line would typically be measured relative to the first hybrid line (i.e., the first hybrid line is the control or reference plant).

4. A plant comprising a recombinant DNA construct: the plant may be assessed or measured relative to a control plant not comprising the recombinant DNA construct but otherwise having a comparable genetic background to the plant (e.g., sharing at least 90%, 91%, 92%, 93%, 94%, 95%, 96%, 97%, 98%, 99%, or 100% identity of nuclear genetic material compared to the plant comprising the recombinant DNA construct.

Methods

Methods include but are not limited to methods for regulating plant flowering time, methods for observing and/or evaluating plant agricultural characteristics, methods for modifying or altering the host endogenous genomic gene, methods for altering the expression and/or activity of endogenous polypeptide, and methods for producing seed.

Methods include but are not limited to the following: Methods are provided for genome modification of a target sequence in the genome of a plant or plant cell, for selecting plants, for gene editing, and for inserting a polynucleotide of interest into the genome of a plant. The methods employ a guide RNA/Cas endonuclease system, wherein the Cas endonuclease is guided by the guide RNA to recognize and optionally introduce a double strand break at a specific target site into the genome of a cell. The guide RNA/Cas endonuclease system provides for an effective system for modifying target sites within the genome of a plant, plant cell or seed. Further provided are methods and compositions employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest.

In one embodiment, a method for modifying a target site in the genome of a plant cell, comprises introducing a guide RNA and a Cas endonuclease into said plant, wherein said guide RNA and Cas endonuclease are capable of forming a complex that enables the Cas endonuclease to introduce a double strand break at said target site.

Further provided is a method for modifying a target site in the genome of a plant cell, the method comprising: a) introducing into a plant cell a guide RNA and a Cas endonuclease, wherein said guide RNA and Cas endonuclease are capable of forming a complex that enables the Cas endonuclease to introduce a double strand break at said target site; and, b) identifying at least one plant cell that has a modification at said target site, wherein the modification includes at least one deletion, insertion or substitution of one or more nucleotides in said target site.

Proteins may be altered in various ways including amino acid substitution, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates containing target sites.

Also provided is a method for editing a nucleotide sequence in the genome of a cell, the method comprising introducing a guide polynucleotide, a Cas endonuclease, and optionally a polynucleotide modification template, into a cell, wherein said guide RNA and Cas endonuclease are capable of forming a complex that enables the Cas endonuclease to introduce a double strand break at target site in the genome at said cell, wherein said polynucleotide modification template comprises at least one nucleotide modification of said nucleotide sequence. The nucleotide sequence in the genome of a cell is selected from the group consisting of a promoter sequence, a terminator sequence, a regulatory element sequence, a splice site, a coding sequence, a polyubiquitination site, an intron site and an intron enhancing motif.

Further provided is a method for editing a promoter sequence in the genome of a cell, the methods comprising introducing a guide polynucleotide, a polynucleotide modification template and at least one Cas endonuclease into a cell, wherein said guide RNA and Cas endonuclease are capable of forming a complex that enables the Cas endonuclease to introduce a double strand break at a target site in the genome of said cell, wherein said polynucleotide modification template comprises at least one nucleotide modification of said nucleotide sequence.

A method for producing a transgenic plant comprising transforming a plant cell with any of the isolated polynucleotides or recombinant DNA constructs of the present disclosure and regenerating a transgenic plant from the transformed plant cell. The disclosure is also directed to the transgenic plant produced by this method, and transgenic seed obtained from this transgenic plant.

A method for isolating a polypeptide of the disclosure from a cell or culture medium of the cell, wherein the cell comprises a recombinant DNA construct comprising a polynucleotide of the disclosure operably linked to at least one regulatory element, and wherein the transformed host cell is grown under conditions that are suitable for expression of the recombinant DNA construct.

A method of altering the level of expression of a polypeptide of the disclosure in a host cell comprising: (a) transforming a host cell with a recombinant DNA construct of the present disclosure; and (b) growing the transformed host cell under conditions that are suitable for expression of the recombinant DNA construct wherein expression of the recombinant DNA construct results in production of altered levels of the polypeptide of the disclosure in the transformed host cell.

A method for producing a modified plant comprising transforming a plant cell with any of the CRISPR-Cas construct of the present disclosure and regenerating a modified plant from the transformed plant cell, wherein, the modified plant and the modified seed obtained by this method may be used in other methods of the present disclosure.

A method for altering the expression level of a polypeptide of the disclosure in a plant comprising: (a) transforming a regenerable plant cell with a CRISPR-Cas construct of the present disclosure; and (b) regenerating a modified plant from the regenerable plant cell after step (a), wherein the plant gene were edited; and (c) growing the transformed plant, wherein the expression of the CRISPR-Cas construct results in production of altered levels of the polypeptide of the disclosure in the transformed plant.

A method of making a plant in which the expression or the activity of an endogenous CMP1 polypeptide is decreased through an introduced genetic modification, when compared to the expression or activity of wild-type CMP1 polypeptide from a control plant, and wherein the plant exhibits early flowering time compared to the control plant, wherein the method comprises the steps of (i) introducing a DNA fragment, deleting a DNA fragment or replacing a DNA fragment, or introducing (ii) one or more nucleotide changes in the genomic region comprising the endogenous CMP1 gene and its promoter, wherein the change is effective for decreasing the expression or the activity of the endogenous CMP1 polypeptide.

A method of producing seed comprising any of the preceding methods, and further comprising obtaining seeds from said progeny plant, wherein said seeds comprise in their genome said recombinant DNA construct.

One embodiment provides a method for delaying flowering time of a plant (e.g., rice) the method comprising increasing the expression of a polynucleotide encoding a polypeptide having at least 90% sequence identity to SEQ ID NO: 7. The increase in expression of the polynucleotide may be mediated by any of the methods described herein using any of the polynucleotides or compositions described herein.

One embodiment provides a method for shortening the flowering time (i.e., early flowering time) of a plant (e.g., rice) the method comprising decreasing the expression of a polynucleotide encoding a polypeptide having at least 90% sequence identity to SEQ ID NO: 7. The decrease in expression of the polynucleotide may be mediated by any of the methods described herein using any of the polynucleotides or compositions described herein.

In some embodiments, the disclosure provides seeds that comprise in their genome the recombinant DNA construct of the disclosure.

The introduction of recombinant DNA constructs of the present disclosure into plants may be carried out by any suitable technique, including but not limited to direct DNA uptake, chemical treatment, electroporation, microinjection, cell fusion, infection, vector mediated DNA transfer, bombardment, or Agrobacterium mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

### Stacking of Traits

Modified plants may comprise a stack of one or more flowering time-regulating polynucleotides disclosed herein with one or more additional polynucleotides resulting in the production or suppression of multiple polypeptide sequences. Modified plants comprising stacks of polynucleotide sequences can be obtained by either or both of traditional breeding methods or through genetic engineering methods. These methods include, but are not limited to, breeding individual lines each comprising a polynucleotide of interest, genome editing, transforming a transgenic plant comprising a gene disclosed herein with a subsequent gene and co-transformation of genes into a single plant cell. As used herein, the term “stacked” includes having the multiple traits present in the same plant (i.e., both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other. A trait, as used herein, refers to the phenotype derived from a particular sequence or groups of sequences. Expression of the sequences can be driven by the same promoter or by different promoters. In certain cases, it may be desirable to introduce a transformation cassette that will suppress the expression of the polynucleotide of interest. This may be combined with any combination of other suppression cassettes or over-expression cassettes to generate the desired combination of traits in the plant. It is further recognized that polynucleotide sequences can be stacked at a desired genomic location using a site-specific recombination system.

### EXAMPLES

### Example 1

### Creation of a Rice Population with an Activation-Tagging Construct

A binary construct that contains four multimerized enhancers elements derived from the Cauliflower Mosaic Virus 35S (CaMV 35S) promoter was used, and the rice activation tagging population was developed from Zhonghua 11 (Oryza sativa L.) which was transformed by Agrobacteria-mediated transformation method as described by Lin and Zhang ((2005) Plant Cell Rep. 23:540-547). Zhonghua 11 was cultivated by the Institute of Crop Sciences, Chinese Academy of Agricultural Sciences. The first batch of seeds used in this research was provided by Beijing Weiming Kaituo Agriculture Biotech Co., Ltd. Calli induced from embryos was transformed with Agrobacteria with the vector. The transgenic lines generated were developed and the transgenic seeds were harvested to form the rice activation tagging population.

### Example 2

### Flowering Trait Observation of the Rice Activation-Tagging Population

The rice plants were planted in the Beijing field (40 °13′N) or in Xinjiang field (40 °34′N) and the phenotype was recorded during the plant growth.

Method:

The rice seeds were sterilized by 800 ppm carbendazol for 8 h at 32° C. and washed 3-5 times with distilled water, then soaked in water for 16 h at 32° C., germinated for 18 h at 35˜37° C. in an incubator. The germinated seeds were planted in a seedbed field, and at 3-leaf stage, the seedlings were transplanted into field. Ten plants from each activation tagged line (ATL) were planted in one row, and ZH11-TC (tissue cultured Zhonghua 11) planted nearby the ATLs in the same block were used as controls.

The rice plants were managed by normal practice using pesticides and fertilizers. Plant phenotypes were observed and recorded during the experiments.

Heading date and maturity date were recorded. The heading date contains the first heading date and the 50% heading date. The first heading date is the date when the first panicle, usually the main stem panicle, heads out the sheath of flag leaf; and the 50% heading date is the date when 50% young panicles head out the sheath of flag leaf for plants in one row. The maturity date is the date when 90% glume, grain spikelet axis or vice glume become yellow from appearance. If the heading date of the ATL plants are earlier than that of the control plants, the ATL line is thought to be early heading plants, and if the heading date of the ATL plants are later than that of the control plants, the ATL line is thought to be late heading plants. The inserted T-DNA may affect the gene in the ATL and some genes may contribute to the regulation of the flowering time of ATL plants.

The plant height, effective panicle number and grain yield per plant were measured. The plant height is the length from the surface of the field to the top of the highest panicle or leaf and is measured before harvest. At the end of the season, all or about six representative plants of each transgenic line were harvested from the middle of the row per line. The panicles first were cut and stored in one bag, and then the stems were cut above the earth and put in another bag. The effective panicle number per plant was obtained by counting, and the grain yield per plant was measured. The plant height, effective panicle number and grain yield data were statistically analyzed using mixed linear model by ASRemI program.

Result:

### 1) AH41120

Forty T2 generation AH41120 and 39 ZH11-TC plants were sowed transplanted in Beijing paddy field and were grown under normal growth conditions and under standard growth practice. On July 21, AH41120 rice plants in four rows exhibited 50% young panicles out the sheath of the flag leaf; while the ZH11-TC rice plants exhibited 50% young panicles on August 20. The result demonstrates that the AH41120 rice plants headed earlier than the control plants by about 30 days. The rice grain yields were measured. As shown in Table 2, the grain yield per AH41120 rice plant was higher than that of control ZH11-TC.

TABLE 2
Flowering trait and grain yield analysis
of AH41120 rice plants (1^{st} experiment)

| | Number | Number | Days to | Grain | | |
|---|---|---|---|---|---|---|
| | of total | of Harvest | 50% heading | yield per | | P |
| ATL ID | plants | plants | date (d) | plant (g) | Diff | value |
| ZH11-TC | 40 | 24 | 65.0 | 36.3 | | |
| AH41120 | 39 | 22 | 95.0 | 38.0 | 1.8 | 0.380 |

Forty AH41120 and 39 ZH11-TC plants were sowed and transplanted in a Beijing paddy field in the next year and the results are shown in Table 3. AH41120 rice plants headed earlier than the control plants by about 19 days. As shown in Table 3, the grain yield per plant of AH41120 rice plant was slightly less than that of ZH11-TC.

TABLE 3
Flowering trait and grain yield analysis
of AH41120 rice plants (2^{nd} experiment)

| | Number | Number | Days to | Grain | | |
|---|---|---|---|---|---|---|
| | of total | of harvest | 50% heading | yield per | | P |
| ATL ID | plants | plants | date (d) | plant (g) | Diff | value |
| ZH11-TC | 40 | 24 | 85.0 | 33.4 | | |
| AH41120 | 39 | 20 | 104.0 | 32.8 | −0.6 | 0.811 |

### 2) AH11081

Two AH11081 rice plants that survived were planted and one rice plant was observed early heading trait by about 21 days. Then AH11081 rice plants were tested under field drought condition in Xinjiang province twice, wherein, a sub-population of AH11081 rice plants demonstrated early heading and early maturity, and 23 of 39 AH11081 rice plants exhibited early heading by about 15 days.

### Example 3

### Flowering Trait Validation of ATL Rice Plants

Optimal production requires the precise regulation of heading date or flowering time, which varies depending on planting location and climate.

To further investigate the flowering trait of ATL rice plants, T2 seeds were planted in different locations or environments: HN (Hainan, 18 °30′N), CS (Changsha, 28 °11′N); BJ (Beijing, 40 °13′N) and NX (Ningxia, 38 °36′N, altitude 1106.3 m).

The method is the same as described in Example 2.

### Results:

### 1) AH41120-CS

Twenty AH41120 rice plants (T-DNA inserted, AH41120-F) were planted in Changsha paddy field, and ZH11-TC and segregated nulls from T-DNA inserted AH41120 (No T-DNA insertion, AH41120-N) were planted nearby and used as control. As shown in Table 4, there is no difference among AH41120-F rice plants and their controls for heading date and maturity date.

TABLE 4
Flowering trait validation of AH41120 rice plants in Changsha field

| | Day to | 50% heading date | | Days to | maturity date | |
|---|---|---|---|---|---|---|
| | 50% heading | Diff with | Diff with | maturity | Diff with | Diff with |
| ATL ID | date (d) | ZH11-TC | AH41120-N | date (d) | ZH11-TC | AH41120-N |
| ZH11-TC | 71.2 | | | 122.0 | | |
| AH41120-N | 71.0 | −0.2 | | 122.0 | 0 | |
| AH41120-F | 71.0 | −0.2 | 0 | 122.0 | 0 | 0 |

### 2) AH41120-BJ

Two batches of seeds were sowed at different times, the second batch was later than the first batch by about 10 days. In these two batches, 20 AH41120-F rice plants were planted in Beijing paddy field, and ZH11-TC and AH41120-N rice plants were planted nearby and used as control. As shown in Table 5, in the first batch, AH41120-F rice plants headed earlier than ZH11-TC and AH41120-N rice plants by 20 and 21 days, respectively; and AH41120-F rice plants matured earlier for 1.5 and 2 days than the control plants. In the second batch, AH41120-F rice plants headed earlier than ZH11-TC and AH41120-N rice plants by 13 and 15 days, respectively; and AH41120-F rice plants exhibited early maturity by about 10.8 and 13 days than the control plants. The grain yield of AH41120-F rice plants was equal to that of ZH11-TC and AH41120-N rice plants (Table 6). These results further demonstrated that AH41120-F were capable of heading earlier and mature earlier than controls in field.

TABLE 5
Flowering trait validation of AH41120 rice plants in Beijing field

| | Days to | 50% heading date | | Days to | maturity date | |
|---|---|---|---|---|---|---|
| | 50% heading | Diff with | Diff with | maturity | Diff with | Diff with |
| ATL ID | date (d) | ZH11-TC | AH41120-N | date (d) | ZH11-TC | AH41120-N |
| ZH11-TC | 98.0 | | | 138.5 | | |
| AH41120-N | 99.0 | 1.0 | | 139.0 | 0.5 | |
| AH41120-F | 78.0 | −20.0 | −21.0 | 137.0 | −1.5 | −2.0 |
| ZH11-TC | 94.2 | | | 132.8 | | |
| AH41120-N | 96.0 | 1.8 | | 135.0 | 2.2 | |
| AH41120-F | 81.0 | −13.2 | −15.0 | 122.0 | −10.8 | −13.0 |

TABLE 6
Grain yield analysis of AH41120 rice plants in Beijing field

| | Grain yield | Diff with | Diff with |
|---|---|---|---|
| ATL ID | per plant (g) | ZH11-TC | AH41120-N |
| ZH11-TC | 38.9 | | |
| AH41120-N | 38.8 | | |
| AH41120-F | 36.7 | −2.2 | −2.1 |

### 3) AH11081-CS

Twenty AH11081 rice plants (T-DNA inserted, AH11081-F) were planted in Changsha paddy field, and ZH11-TC and segregated nulls from T-DNA inserted AH11081 rice plants (No T-DNA insertion, AH11081-N) were planted nearby and used as control. As shown in Table 7, AH11081-F rice plants headed 7 and 8 days earlier than ZH11-TC and AH11081-N rice plants, respectively. These results demonstrated that AH11081-F can head earlier and mature earlier than controls in the field.

TABLE 7
Flowering trait validation of AH11081 rice plants in Changsha field

| | Day to | 50% heading date | | Days to | maturity date | |
|---|---|---|---|---|---|---|
| | 50% heading | Diff with | Diff with | maturity | Diff with | Diff with |
| ATL ID | date (d) | ZH11-TC | AH11081-N | date (d) | ZH11-TC | AH11081-N |
| ZH11-TC | 71.6 | | | 116.6 | | |
| AH11081-N | 73.0 | 1.4 | | 118.0 | 1.4 | |
| AH11081-F | 65.0 | −6.6 | −8.0 | 110.0 | −6.6 | −8.0 |

### 4) AH11081-BJ

Twenty AH11081-F were planted in Beijing paddy field, and ZH11-TC and AH11081-N were planted nearby and used as control. As shown in Table 8, AH11081-F rice plants headed 17 and 20 days earlier than ZH11-TC and AH11081-N rice plants, respectively. Table 9 shows that the grain yield per plant of AH11081-F rice plant was equal to that of ZH11-TC and greater than AH11081-F rice plants. These results further demonstrated that AH11081-F rice plants can head earlier and mature earlier than controls in field.

The data in Example 2 and Example 3 further demonstrated that AH11081 rice plants can head earlier and mature earlier than controls in different locations (Xinjiang, Changsha and Beijing).

TABLE 8
Flowering trait validation of AH11081 rice plants in Beijing field

| | Days to | 50% heading date | | Days to | maturity date | |
|---|---|---|---|---|---|---|
| | 50% heading | Diff with | Diff with | maturity | Diff with | Diff with |
| ATL ID | date (d) | ZH11-TC | AH11081-N | date (d) | ZH11-TC | AH41120-N |
| ZH11-TC | 100.9 | | | 156.0 | | |
| AH11081-N | 104.0 | | | 156.0 | | |
| AH11081-F | 84.0 | −16.9 | −20.0 | 141.0 | −15.0 | −15 |

TABLE 9
Grain yield analysis of AH11081 rice plants in Beijing field

| | Grain yield | Diff with | Diff with |
|---|---|---|---|
| ATL ID | per plant (g) | ZH11-TC | AH11081-N |
| ZH11-TC | 50.5 | | |
| AH11081-N | 40.4 | | |
| AH11081-F | 49.3 | −0.2 | −10.1 |

These results demonstrated that AH41120 and AH11081 rice plants showed early heading date or flowering time in different locations such as Changsha (28 °11′N), Beijing (40 °13′N), Ningxia (38 °36′N, altitude 1106.3 m) and Xinjiang (40 °34′N).

In light of these results, the gene(s) which contributed to the early heading date or flowering time of Line AH41120 and AH11081 were isolated.

### Example 4

### Identification of Activation-Tagged Genes

Genes flanking the T-DNA insertion locus in AH41120 and AH11081 were identified. Genomic DNA was isolated from leaf tissues of the AH41120 and AH11081 lines using the CTAB method (Murray, M. G. and W. F. Thompson. (1980) Nucleic Acids Res. 8: 4321-4326). The flanking sequences of T-DNA insertion locus were obtained by molecular technology.

One T-DNA inserted in chromosome 7 of AH41120 rice plans (MSU7.0 http://rice.plantbiology.msu.edu/index.shtml), and one T-DNA inserted in chromosome 7 of AH11081 rice plants. The nucleotide sequences flanking the T-DNA insertion locus in AH41120 were shown in SEQ ID NO: 1 and 2, and the nucleotide sequences flanking the T-DNA insertion locus in AH11081 were shown in SEQ ID NO: 3 and 4. Further analysis shows that the T-DNA inserted at about 3500 bp upstreaming the coding sequence of LOC_Os07g15770 in AH41120 rice plants; and the T-DNA inserted in the coding region of LOC_Os07g15770, the inserting site is at about 10 bp following the initiation codon in AH11081 rice plants, so the gene at rice gene locus LOC_Os07g15770 was isolated.

The rice gene at locus LOC_Os07g15770.1 encodes OsCMP1, a CMP1 polypeptide. This polypeptide is annotated as “CCT motif family protein, expressed” in TIGR (the internet at plant biology msu.edu/index.shtml).

### Example 5

### Flowering Time-Regulating Gene Cloning and Over-Expression Vector Construction

Primers were designed for cloning rice flowering time-regulating gene (OsCMP1, LOC_Os07g15770). The OsCMP1 gDNA was cloned from genomic DNA of Zhonghua 11 plant using conventional methods and the following primers:

gcl-3433:
(SEQ ID NO: 9)
5′-ATGGGGATGGCCAATGAGGAG-3′
gcl-3434:
(SEQ ID NO: 10)
5′-ATCTATCTGAACCATTGTCCAAGCTC-3′.

The PCR amplified product (2786 bp in length) was extracted after agarose gel electrophoresis using a column kit and then ligated with TA cloning vectors. The sequence and orientation in the construct were confirmed by sequencing. The gene was cloned into plant binary construct DP0158 (pCAMBIA1300-DsRed). The cloned nucleotide sequence in construct of DP2300 and coding sequence of OsCMP1 are provided as SEQ ID NO: 5 and 6, the encoded amino acid sequence of OsCMP1 is shown in SEQ ID NO: 7.

### Example 6

### Generation Rice Plants with Increased Gene Expression

The over-expression vector and empty vector (DP0158) were transformed into Zhonghua 11 (Oryza sativa L.) by Agrobacteria-mediated as described by Lin and Zhang (Plant Cell Rep. 23: 540-547 (2005)). The transgenic seedlings (T0) generated in transformation laboratory were transplanted in the field to get T1 seeds. The T1 and T2 seeds were stored at 4° C. The over-expression vectors contain DsRED and HYG genes. T1 and T2 seeds which showed red color under green fluorescent light were transgenic seeds and were used in the following flowering trait assays.

Gene Expression Analysis in Transgenic Rice Plants:

Gene expression levels in the transgenic rice plants were analyzed by a standard real-time RT-PCR procedure. EF1a gene was used as an internal control to show that the amplification and loading of samples from the transgenic rice and control plant were similar. The expression level was normalized based on the EF1a mRNA levels.

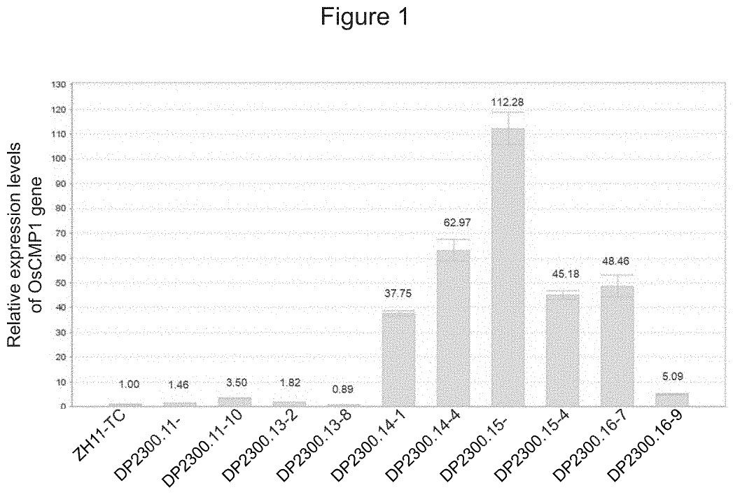

OsCMP1 gene expression levels in the DP2300 rice plants were detected using the primers of SEQ ID NOs: 11 and 12. mRNA was extracted from the top second leaf of T1 generation seedlings which were at heading stage. As shown in FIG. 1, the expression level in ZH11-TC rice is set at 1.00, and OsCMBP1 over-expressed in the transgenic lines, however, the expression levels were different in different plants from the same line. These results indicate that there is segregation for the OsCMP1 gene at different generation.

DP2300-F1:
(SEQ ID NO: 11)
5′-ACGAGAAGCAAATCCGGTAC-3′
DP2300-R1:
(SEQ ID NO: 12)
5′-TGATCAGGTTCTTTGGCGAAG-3′

### Example 7

### Flowering Trait Observation for the OsCMP1 Over-Expression Rice Plants

Forty transgenic seedlings (T0, DP2300) generated in the transformation laboratory were transplanted in the field to get T1 seeds in early August in Beijing (40 °13′N). The day length gets shorter and the nights get longer from August to November. The longer night/shorter day promote rice early heading. The temperature began to decrease from the late October, so electric heaters were used to heat the cage in which the transgenic rice plants planted at the late October to November to ensure suitable temperature and good grain filling. ZH11-TC and other transgenic rice plants planted at the same time were harvested at the early November, however, there was no panicle headed out from the OsCMP1 transgenic rice plants at the middle of November.

The T0 stubbles were transplanted into Hainan field (18 °30′N) in late November. ZH11-TC and other transgenic rice plants planted at the same time were harvested at the late February. Five OsCMP1 transgenic rice plants showed 5 to 10 days later heading date than ZH11-TC and other transgenic rice plants planted at the same time; two OsCMP1 transgenic rice plants showed 62 days, six OsCMP1 transgenic rice plants showed 90 days later heading date; and the other OsCMP1 transgenic rice plants did not head out. Finally, T1 seeds from five transgenic plants were obtained.

The T0 OsCMP1 transgenic rice plants, planted in Beijing and Hainan fields, showed high and thick stem. These OsCMP1 transgenic rice plants were about 20 to 30 cm higher than ZH11-TC and other transgenic rice plants planted nearby in the Beijing field.

These results showed that OsCMP1 over-expressed rice plants head late or no heading at different environments. Over-expression of OsCMP1 gene affects the heading date or flowering time, and OsCMP1 gene regulates the heading date or flowering time and height of rice plants.

### Example 8

### Flowering Trait Validation of OsCMP1 Over-Expression Rice Plants

To further investigate the flowering trait of OsCMP1 transgenic rice plants (DP2300) and to investigate whether the temperature or photoperiod affect the heading date or flowering time in rice, T1 and T2 seeds were planted in different locations or environments: HN (Hainan, 18 °30′N), CS (Changsha, 28 °11′N), and BJ (Beijing, 40 °13′N).

The experimental method is the same as described in Example 2.

### Results:

### 1) DP2300-CS

Five OsCMP1 over-expression rice lines were tested in Changsha field, ZH11-TC rice plants were planted nearby and used as control. As shown in Table 10, all transgenic rice plants headed later than ZH11-TC. DP2300.11, DP2300.13 and DP2300.16 rice plants headed later by 4 days, 5 days and 5 days than ZH11-TC control, respectively. DP2300.14 and DP2300.15 rice plants headed later by 25 days and 50 days than ZH11-TC control, respectively. These results demonstrate that OsCMP1 over-expression rice plants headed later than control plant at 28°N. The more expression of OsCMP1 gene, the later heading date. The later headed rice plants except DP2300.14 and DP2300.15 matured before harvesting. The maturity dates were similar to that of ZH11-TC plants.

TABLE 10
Flowering trait validation of OsCMP1 over-
expression rice plants in Changsha field

| | Days to | Diff of | Days to | Diff of |
|---|---|---|---|---|
| | heading | heading | maturity | maturity |
| Line ID | date (d) | date | date (d) | Date |
| ZH11-TC | 71.0 | | 115.0 | |
| DP2300.11 | 75.0 | 4.0 | 116.0 | 1.0 |
| DP2300.13 | 76.0 | 5.0 | 116.0 | 1.0 |
| DP2300.14 | 121.0 | 50.0 | | |
| DP2300.15 | 96.0 | 25.0 | | |
| DP2300.16 | 76.0 | 5.0 | 116.0 | 1.0 |

### 2) DP2300-BJ

The same five OsCMP1 over-expression rice lines were tested in the Beijing field, ZH11-TC rice plants were planted nearby and used as control. Ten plants for each transgenic line were planted. As shown in Table 11, all the DP2300.11 and DP2300.13 rice plants headed 3 days and 1 day later than ZH11-TC control. Part of DP2300.14, DP2300.15 and DP2300.16 rice plants headed later than ZH11-TC control. Four DP2300.14 rice plants, one DP2300.15 rice plant and three DP2300.16 rice plants didn't head before harvesting. The late headed rice plants except DP2300.14 matured before harvesting. The maturity dates were similar to that of ZH11-TC plants. These results further demonstrate that OsCMP1 over-expression rice plants headed later or no heading at different environments.

TABLE 11
Flowering trait validation of OsCMP1 over-
expression rice plants in Beijing field

| | Days to | Diff of | Days to | Diff of |
|---|---|---|---|---|
| | heading | heading | maturity | maturity |
| Line ID | date (d) | date | date (d) | Date |
| ZH11-TC | 98.0 | | 138.5 | |
| DP2300.11 | 101.0 | 3.0 | 139.0 | 0.5 |
| DP2300.13 | 99.0 | 1.0 | 139.0 | 0.5 |
| DP2300.14 | 139.0 | 41.0 | 172.0 | 33.5 |
| DP2300.15 | 102.0 | 4.0 | 139.0 | 0.5 |
| DP2300.16 | 100.0 | 2.0 | 139.0 | 0.5 |

### Example 9

### Design of SCI RNA Sequence

Target genomic sequences are analyzed using available tools to generate candidate sgRNA sequences. The sgRNA sequences can also be generated by web-tools including, but not limited to, the web site cbi.hzau.edu.cn/crispr/and CRISPR-PLANT, available online.

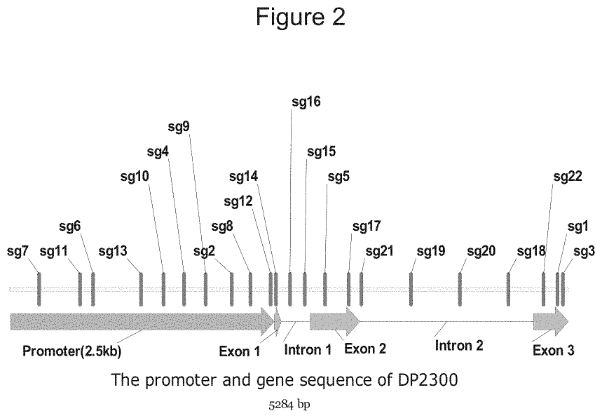

In this application, the OsCMP1 promoter and gene sequence (SEQ ID NO: 8 and SEQ ID NO: 5) were analyzed to generate the sgRNA sequences. The OsCMP1 promoter and gene sequence includes promoter, exon, intron, 5′-UTR, and 3′-UTR, and many sgRNA sequences were generated. 22 sgRNA sequences were selected and the distribution is shown in FIG. 2. The sgRNA sequences are provided in SEQ ID NOs: 18-39.

### Example 10

### Construction of CRIPSR-Cas constructs for OsCMP1 gene

In the CRISPR-Cas9 system, maize Ubi promoter (SEQ ID NO: 13) drives the optimized coding sequence (SEQ ID NO: 14) of Cas9 protein; CaMV35S 3′-UTR (SEQ ID NO: 15) improves the expression level of Cas9 protein; and rice U6 promoter (SEQ ID NO: 16) drives the expression of gRNA (gRNA scaffold, SEQ ID NO: 17).

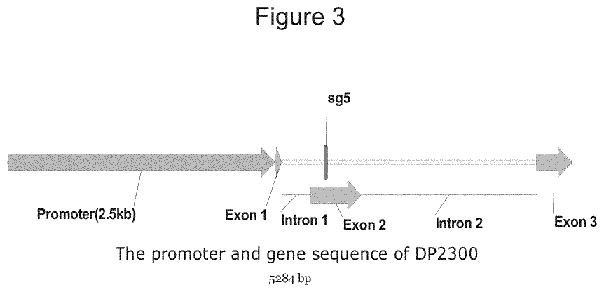

One sgRNA can be used to make the genome editing construct (FIG. 3.); the sgRNA can be selected from any region of the fragment such as the promoter, exon, intron and UTR. The single sgRNA can guide the Cas9 enzyme to the target region and generate the double strand break at the target DNA sequence, non-homologous end-joining (NHEJ) and homology directed repair (HDR) will be triggered, and it often induces random insertion, deletion and substitution at the target site. This edit, for example, can remove an expression element in the promoter region of OsCMP1 to reduce the mRNA levels or if the edit is in the coding region can result in a structural change in the OsCMP1 polypeptide that may result in reduced activity of the OsCMP1 protein.

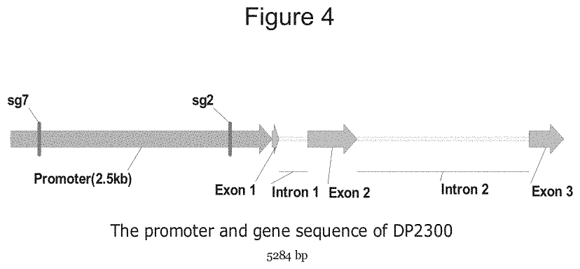

Two sgRNAs can be used to make the genome editing construct (FIG. 4.); two or more sgRNAs can be selected from any region of the fragment such as promoter, exon, intron and UTR. This construct can lead to fragment deletion, point mutation (small insertion, deletion and substitution).

Table 12 shows the primer sequence, target position and the specific strand. For the construct DP2855, one sgRNA was used. The target primers first annealed to form short double strand fragment, then the fragment was inserted in pHSG396GW-URS-UC-mpCas9&U6-DsRed (improved vector from VK005-01, which was bought from Beijing Veiwsolid Biotech company). The elements in the cloning vector pHSG396GW-URS-UC-mpCas9&U6-DsRed were shown in SEQ ID NO: 13, SEQ ID NO: 14, SEQ ID NO: 15, SEQ ID NO: 16 and SEQ ID NO: 17. After confirming the nucleotide sequence of the gRNA fragment, the gRNA fragment was ligated with the expression vector PCAMBIA1300DsRed-GW-Adv.ccdB. For the construct having two sgRNAs, the different primers should first anneal to form the double strand fragments, then the two gRNA fragments stacked together and inserted in the cloning vector, and then were inserted in the expression vector to form DP2925. The expected cleaving site was shown in FIG. 2.

The sgRNA(s) in the construct DP2855 and DP2925 targets to the genomic region containing OsCMP1 gene.

TABLE 12
Primers for constructing CRISPR/Cas9
constructs for OsCMP1 gene editing

| Con- | Target | | | Target site | SEQ |
|---|---|---|---|---|---|
| struct | site | | Strand | primer | ID |
| ID | ID | Target position | (+/−) | sequence | NO: |
| DP2855 | gRNA5 | Chr7: | + | 5′-CCGATCCCCT | 22 |
| | | 9154687-9154706 | | GGCACGCACT-3′ | |
| DP2925 | gRNA5 | Chr7: | + | 5′-CCGATCCCCT | 22 |
| | | 9154687-9154706 | | GGCACGCACT-3′ | |
| | gRNA1 | Chr7: | + | 5′-TGGCGAAGCG | 18 |
| | | 9152485-9152504 | | ACCTCTCACT-3′ | |

### Example 11

Transformation to Obtain Genome Edited Rice The CRISPR-Cas9 constructs for OsCMP1 gene were transformed into the Zhonghua 11 and Daohuaxiang 2, by an Agrobacteria-mediated method as described by Lin and Zhang ((2005) Plant Cell Rep. 23:540-547). The transformed seedlings (T0) generated in the transformation laboratory were first validated by PCR and sequencing, and then were planted in the field to get T1 seeds. The T1 and T2 seeds were stored at 4° C. The obtained Zhonghua 11 rice plants were labeled as DP2855, DP2925; and the obtained Daohuaxiang 2 rice plants were labeled as RL2855, RL2925.

### Example 12

### Identification the Cleavage Sites and the Modifications of OsCMP1 Gene in Rice Plants

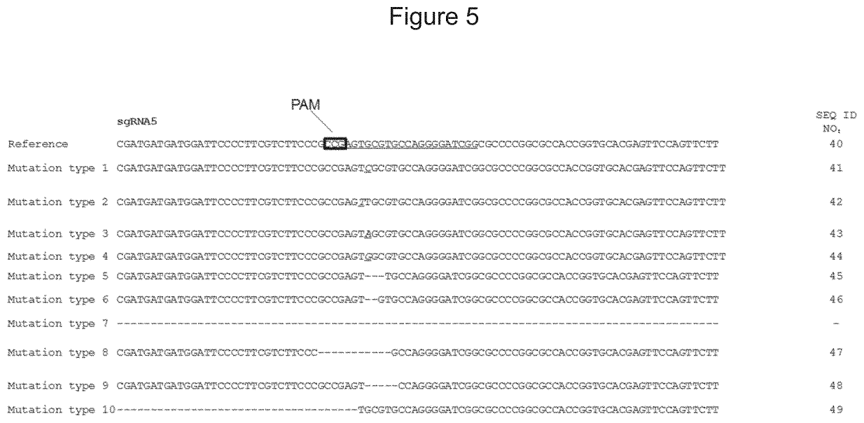

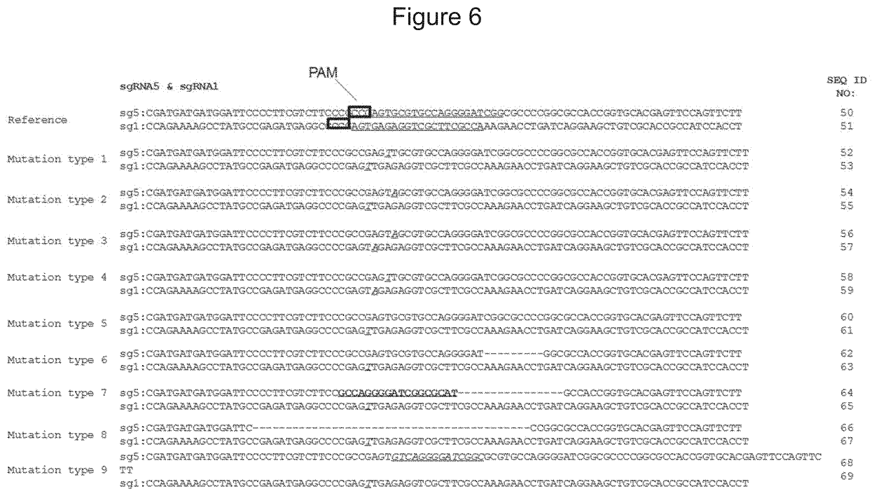

The primers were designed to amplify the target sequence near the genome editing target sites using the genomic DNA of the transformed seedlings as template. The amplified target sequences were sequenced to confirm the editing results, which are shown in FIGS. 5 and 6. Modifications such as insertion of at least one nucleotide, deletion of at least one nucleotide, replacement of at least one nucleotide were produced, which resulted early termination of the coding sequence, translation shift and/or deletion of at least one amino acid residues.

As shown in FIG. 5, there are ten modifications produced at the expected site in DP2855 rice plants (ZH11). One nucleotide inserted at the expect site in mutation types 1 to 4 and resulted in translation shift, and thereby resulted in early stop of the ORF. The predicted translated polypeptide has 104 amino acids. Three nucleotides were deleted in mutation type 5 and resulted in one amino acid missing in the predicted translated polypeptide. Two nucleotides were deleted in mutation type 6 and resulted in translation shift, and thereby resulted in early stops of the ORF. More nucleotides were deleted in mutation types 7 to 10 and resulted in early stops in the ORF.

As shown in FIG. 6, there are about nine modifications produced at the expected sites in DP2925 rice plants (ZH11). One nucleotide inserted at the two expected sites in mutation types 1 to 4 respectively, and resulted in translation shift, and thereby resulted in early stop of the ORF. These mutations produced the similar polypeptides as that in DP2855 rice plants (mutation types 1 to 4). One nucleotide inserted in the second expected site in mutation types 5 and 6 and resulted in early stops of the ORF and produced polypeptides with more than 259 amino acids. The mutation types 7 to 9 also resulted in early stops of the ORF and produced polypeptides with more than 199 amino acids.

The mutations in DP2855 rice plants and DP2925 rice plants resulted in early termination of the translation of OsCMP1 coding sequence, the predicted polypeptides don't have the domain of CCT superfamily domain or have incomplete CCT superfamily domain, which further affect the length and the activity of the translated polypeptides.

Similar mutations occurred in RL2855, RL2925 rice plants.

The genome edited homozygous rice plants were used in the following functional tests.

### Example 13

### Flowering Trait Observation of OsCMP1 Modified Rice Plants

### Results:

### 1) T0 Generation ZH11

### DP2855-BJ

The transformed ZH11 seedlings (T0, DP2855) generated in transformation laboratory were transplanted in the field to get T1 seeds in Beijing (40 °13′N). The phenotype was recorded during the plant growth. The first heading date which is date when the first panicle heads out the sheath of flag leaf of one plant for about 2 cm is used. The days from transplanting to the first heading date were used. DP2855H, DP2855P and DP2855N represent homozygous, hemizygous and genome editing negative ZH11 rice plants at the expect targeting sites, respectively. As shown in Table 13, the average days to first heading date of DP2855 homozygous rice plants were about 10 days less than that of ZH11-TC rice plants and the average days to first heading date of DP2855 hemizygous rice plants were about 6 days less than that of ZH11-TC rice plants. The average days to first heading date of DP2855 negative rice plants were equal to that of ZH11-TC rice plants. DP2855 homozygous and hemizygous rice plants headed earlier than ZH11-TC in Beijing at T0 generation.

TABLE 13
Flowering trait of OsCMP1 gene edited (DP2855)
ZH11 rice plants at T0 generation in Beijing

| | | Average days | Diff of first heading |
|---|---|---|---|
| | Plant | to first heading | date compared |
| Construct ID | number | date (d) | with ZH11-TC |
| ZH11-TC | 20 | 57.0 ± 1.9 | |
| DP2855N | 10 | 58.6 ± 1.8 | 1.6 |
| DP2855H | 15 | 47.3 ± 3.4 | −9.7 |
| DP2855P | 26 | 51.5 ± 6.6 | −5.5 |

### DP2925-BJ

The transformed ZH11 seedlings (T0, DP2925) generated in the transformation laboratory were transplanted in the field to get T1 seeds in Beijing (40 °13′N). The phenotype was recorded during the plant growth. The first heading date is used. DP2925H, DP2925P and DP2925N represent homozygous, hemizygous and genome editing negative ZH11 rice plants at the expect targeting sites, respectively. As shown in Table 14, the average days to first heading date of DP2925 homozygous rice plants were about 12 days less than that of ZH11-TC rice plants and the average days to first heading date of DP2925 hemizygous rice plants were about 8 days less than that of ZH11-TC rice plants. The average days to first heading date of DP2925 negative rice plants were more that of ZH11-TC rice plants. DP2925 homozygous and hemizygous rice plants headed earlier than ZH11-TC in Beijing at T0 generation.

TABLE 14
Flowering trait of OsCMP1 gene edited (DP2925)
ZH11 rice plants at T0 generation in Beijing

| | | Average days | Diff of first heading |
|---|---|---|---|
| | Plant | to first heading | date compared |
| Construct ID | number | date (d) | with ZH11-TC |
| ZH11-TC | 20 | 57.0 ± 1.9 | |
| DP2925N | 10 | 60.4 ± 2.2 | 3.0 |
| DP2925H | 17 | 45.0 ± 4.1 | −12.0 |
| DP2925P | 32 | 49.0 ± 4.7 | −8.0 |

### 2) T0 Generation Daohuaxiang 2

### RL2855-HN

The first batch of transformed Daohuaxiang 2 seedlings (T0, RL2855) generated in the transformation laboratory were transplanted in the field to get T1 seeds in late October in Hainan (18 °30′N). The phenotype was recorded during plant growth. The first heading date is used. RL2855H, RL2855P and RL2855N represent homozygous, hemizygous and genome editing negative Daohuaxiang 2 rice plants at the expected targeting sites, respectively. The day length gets shorter and the night length get longer from late September to middle December and the day length on December 20 was shortest in Hainan. The longer night/shorter day will promote rice early heading. As shown in Table 15, the average days to first heading date of RL2855 homozygous rice plants were about 13 days less than that of RL2855 negative rice plants and the average days to first heading date of RL2855 hemizygous rice plants were about 10 days less than that of RL2855 negative rice plants. These results indicate that RL2855 homozygous and hemizygous rice plants headed earlier than its negative rice plants in Hainan at T0 generation.

TABLE 15
Flowering trait of OsCMP1 gene edited (RL2855) Daohuaxiang
2 rice plants at T0 generation in Hainan (1^{st} experiment)

| | | Average days | Diff of first heading |
|---|---|---|---|
| | Plant | to first heading | date compared |
| Construct ID | number | date (d) | with RL2855N |
| RL2855N | 4 | 47.5 ± 4.2 | |
| RL2855H | 2 | 35.0 ± 4.2 | −12.5 |
| RL2855P | 4 | 38.0 ± 5.0 | −9.5 |

The second batch of transformed Daohuaxiang 2 seedlings (T0, RL2855) generated in transformation laboratory were transplanted in the field to get T1 seeds in early January in Hainan (18 °30′N). The phenotype was recorded during plant growth. The day length gets longer, and the night length get shorter from late December to middle March in Hainan. The longer day/shorter night will promote rice late heading. As shown in Table 16, compared with RL2855 negative rice plants, the average days to first heading date of RL2855 homozygous rice plants were about 6 days less, and the average days to first heading date of RL2855 hemizygous rice plants were about 4 days less. These results also indicate that RL2855 homozygous and hemizygous rice plants headed earlier than its negative rice plants in Hainan at T0 generation; and the photoperiod affect the heading date or flowering time trait.

TABLE 16
Flowering trait of OsCMP1 gene edited (RL2855) Daohuaxiang
2 rice plants at T0 generation in Hainan (2^{nd} experiment)

| | | Average days | Diff of first heading |
|---|---|---|---|
| | Plant | to first heading | date compared |
| Construct ID | number | date (d) | with RL2855N |
| RL2855N | 2 | 45.0 ± 0.0 | |
| RL2855H | 11 | 39.4 ± 2.4 | −5.6 |
| RL2855P | 54 | 40.7 ± 4.1 | −4.3 |

### RL2925-HN

The transformed Daohuaxiang 2 seedlings (T0, RL2925) generated in the transformation laboratory were transplanted in the field to get T1 seeds in early November in Hainan (18 °30′N). The phenotype was recorded during plant growth. The first heading date is used. RL2925H, RL2925P and RL2925N represent homozygous, hemizygous and genome editing negative Daohuaxiang 2 rice plants at the expected targeting sites, respectively. The day length gets shorter and the night length get longer from late September to middle December and the day length on December 20 was shortest in Hainan. The longer night/shorter day will promote rice early heading. As shown in Table 17, the average days to first heading date of RL2925 homozygous rice plants and RL2925 hemizygous rice plants were about 5 days less than that of RL2925 negative rice plants. These results indicate that RL2925 homozygous and hemizygous rice plants headed earlier than its negative rice plants in Hainan at T0 generation.

TABLE 17
Flowering trait of OsCMP1 gene edited (RL2925) Daohuaxiang
2 rice plants at T0 generation in Hainan

| | | Average days | Diff of first heading |
|---|---|---|---|
| | Plant | to first heading | date compared |
| Construct ID | number | date (d) | with RL2925N |
| RL2925N | 2 | 39.2 ± 3.9 | |
| RL2925H | 4 | 34.2 ± 2.2 | −5.0 |
| RL2925P | 9 | 34.0 ± 2.9 | −5.2 |

### Example 14

### Flowering Trait Validation of OsCMP1 Modified Rice

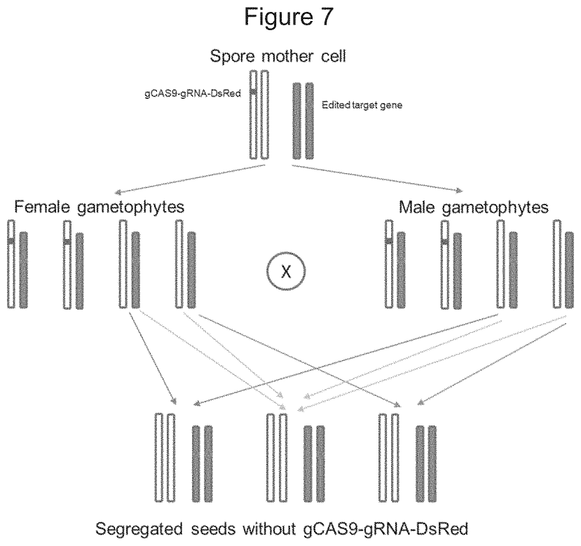

T1 and T2 seeds were obtained from the transformed T0 plants. Because the T-DNA randomly inserts in the 12 chromosomes of rice cell, the T-DNA insertion sites and the target sites may locate on different chromosomes or are far away each other even if located on the same chromosome. The T-DNA is separated from the target sites during germ cell formation from somatic cell by meiosis, to get normal seeds without gCAS9-gRNA-DsRed (FIG. 7.). The seeds were planted and identified by molecular technology. Some mutations are described in Example 12. The character “B” in the line ID indicates normal seeds.

The experimental method is the same as that described in Example 2.

### Results:

### 1) ZH11 Plants

### T1 Generation DP2855-HN

Six homologous T1 generation OsCMP1 modified rice lines (DP2855) were planted, and the ZH11-TC and the genome edited negative rice plants (DP2855N) which went through the transformation process and have the wild-type (un-mutated) OsCMP1 gene were used as controls. One nucleotide “T” inserted in the expected target site in these six OsCMP1 modified rice plants, and resulted in translation shift, thereby resulting in early stops of the ORF. The mutation type in these six OsCMP1 lines belong to type 2 in FIG. 5, and the predicted translated polypeptide has 104 amino acids. These plants were sowed in November, and transplanted in the paddy field in December. The days from sowing to 50% heading date for the ZH11-TC control was 70.5 days, and for DP2855N was 67 days. DP2855H rice plants exhibited about 9 days and 5 days earlier heading date than ZH11-TC and DP2855N rice plants at construct level, respectively. All the DP2855H rice lines exhibited an earlier heading date than ZH11-TC and DP2855N rice plants. As shown in Table 18, all the genome edited rice plants exhibited an earlier maturity date than the controls.

TABLE 18
Flowering trait analysis of OsCMP1 gene edited (DP2855)
ZH11 rice plants at T1 generation in Hainan

| | Days to | | | Days to | | |
|---|---|---|---|---|---|---|
| | 50% heading | Diff of heading date | | maturity | Diff of maturity date | |
| Line ID | date (d) | ZH11-TC | DP2855N | date (d) | ZH11-TC | DP2855N |
| DP2855H | 62.0 | −8.5 | −5.2 | 107.2 | −2.8 | −2.2 |
| (Construct) | | | | | | |
| ZH11-TC | 70.5 | | | 110.0 | | |
| DP2855N | 67.2 | | | 109.4 | | |
| DP2855H.05B | 61.0 | −9.5 | −6.2 | 106.0 | −4.0 | −3.4 |
| DP2855H.06B | 61.0 | −9.5 | −6.2 | 106.0 | −4.0 | −3.4 |
| DP2855H.07B | 63.0 | −7.5 | −4.2 | 108.0 | −2.0 | −1.4 |
| DP2855H.09B | 63.0 | −7.5 | −4.2 | 108.0 | −2.0 | −1.4 |
| DP2855H.11B | 61.0 | −9.5 | −6.2 | 107.0 | −3.0 | −2.4 |
| DP2855H.15B | 63.0 | −7.5 | −4.2 | 108.0 | −2.0 | −1.4 |

### T1 Generation DP2855-CS

Two homologous T1 generation OsCMP1 modified rice lines (DP2855) were planted in a Changsha field, ZH11-WT and DP2855N rice plants were used as controls. One nucleotide “T” inserted in the expected target site in these two OsCMP1 modified rice plants, and resulted in a translation shift, thereby resulting in early stop of the ORF. The mutation type belongs to type 2 in FIG. 5, and the predicted translated polypeptide has 104 amino acids. These plants were sowed in May and transplanted in the paddy field in June. Twenty plants from each line were planted in two rows. The days from sowing to 50% heading date for the ZH11-WT control and DP2855N were 76 days. These two DP2855H rice lines exhibited about 10 days earlier heading date than ZH11-WT and DP2855N rice plants and exhibited early maturity date than the controls (Table 19).

TABLE 19
Flowering trait analysis of OsCMP1 gene edited (DP2855)
ZH11 rice plants at T1 generation in Changsha

| | Days to | | | Days to | | |
|---|---|---|---|---|---|---|
| | 50% heading | Diff of heading date | | maturity | Diff of maturity date | |
| Line ID | date (d) | ZH11-WT | DP2855N | date (d) | ZH11-WT | DP2855N |
| ZH11-WT | 76.0 | | | 121.0 | | |
| DP2855N | 76.0 | | | 121.0 | | |
| DP2855H.08B | 66.0 | −10.0 | −10.0 | 111.0 | −10.0 | −10.0 |
| DP2855H.11B | 66.0 | −10.0 | −10.0 | 111.0 | −10.0 | −10.0 |

### T1 Generation DP2855-BJ

The same two homologous T1 generation OsCMP1 modified rice lines (DP2855) were planted in a Beijing field, and the ZH11-WT and DP2855N rice plants were used as controls. These plants were sowed in May and transplanted in the paddy field in June. Twenty plants from each line were planted in two rows. The days from sowing to 50% heading date for the ZH11-WT control and DP2855N were 92 days. These two DP2855H rice lines exhibited about 21 days earlier heading date than ZH11-WT and DP2855N rice plants and exhibited an early maturity date than the controls (Table 20).

TABLE 20
Flowering trait analysis of OsCMP1 gene edited (DP2855)
ZH11 rice plants at T1 generation in Beijing

| | Days to | | | Days to | | |
|---|---|---|---|---|---|---|
| | 50% heading | Diff of heading date | | maturity | Diff of maturity date | |
| Line ID | date (d) | ZH11-WT | DP2855N | date (d) | ZH11-WT | DP2855N |
| ZH11-WT | 91.8 | | | 139.0 | | |
| DP2855N | 91.5 | | | 139.0 | | |
| DP2855H.08B | 71.0 | −20.8 | −20.5 | 122.0 | −17.0 | −17.0 |
| DP2855H.11B | 71.0 | −20.8 | −20.5 | 122.0 | −17.0 | −17.0 |

### T2 Generation DP2855-CS

Ten homologous T2 generation OsCMP1 modified rice lines (DP2855) were planted in a Changsha field, and the ZH11-WT and DP2855N rice plants were used as controls. One nucleotide “T, A, C, G” inserted in the expected target site in the OsCMP1 modified rice plants, and resulted in translation shift, thereby resulting in early stop of the ORF. The mutations are shown in types 1 to 4 in FIG. 5, and the predicted translated polypeptide has 104 amino acids. These plants were sowed in May and transplanted in the paddy field in June. Twenty plants from each line were planted in two rows. As shown in Table 21, the days from sowing to 50% heading date for the ZH11-WT control and DP2855N were about 76 days. The genome edited DP2855H rice plants exhibited about 8 days earlier heading date than ZH11-WT and DP2855N rice plants at the construct level and exhibited an early maturity date than the controls.

TABLE 21
Flowering trait analysis of OsCMP1 gene edited (DP2855)
ZH11 rice plants at T2 generation in Changsha

| | Days to | | | Days to | | |
|---|---|---|---|---|---|---|
| | 50% heading | Diff of heading date | | maturity | Diff of maturity date | |
| Line ID | date (d) | ZH11-WT | DP2855N | date (d) | ZH11-WT | DP2855N |
| DP2855H | 67.7 | −8.3 | −7.6 | 112.7 | −8.3 | −7.6 |
| (Construct) | | | | | | |
| ZH11-WT | 76.0 | | | 121.0 | | |
| DP2855N | 75.3 | | | 120.3 | | |
| DP2855H.02B | 67.5 | −8.5 | −7.8 | 112.5 | −8.5 | −7.8 |
| DP2855H.05B | 66.5 | −9.5 | −8.8 | 111.5 | −9.5 | −8.8 |
| DP2855H.06B | 67.5 | −8.5 | −7.8 | 112.5 | −8.5 | −7.8 |
| DP2855H.07B | 68.0 | −8.0 | −7.3 | 113.0 | −8.0 | −7.3 |
| DP2855H.08B | 67.5 | −8.5 | −7.8 | 112.5 | −8.5 | −7.8 |
| DP2855H.09B | 67.0 | −9.0 | −8.3 | 112.0 | −9.0 | −8.3 |
| DP2855H.10B | 68.5 | −7.5 | −6.8 | 113.5 | −7.5 | −6.8 |
| DP2855H.11B | 67.5 | −8.5 | −7.8 | 112.5 | −8.5 | −7.8 |
| DP2855H.12B | 68.0 | −8.0 | −7.3 | 113.0 | −8.0 | −7.3 |
| DP2855H.14B | 69.0 | −7.0 | −6.3 | 114.0 | −7.0 | −6.3 |

### T2 Generation DP2855-BJ

The same ten homologous T2 generation OsCMP1 modified rice lines (DP2855) were planted in a Beijing field, and the ZH11-WT and DP2855N rice plants were used as controls. These plants were sowed in May and transplanted in the paddy field in June. Twenty plants from each line were planted in two rows. As shown in Table 22, the days from sowing to 50% heading date for the ZH11-WT control and DP2855N were about 108 days. The genome edited DP2855H rice plants exhibited about 18 days earlier heading date than ZH11-WT and DP2855N rice plants at the construct level, and also exhibited an early maturity date of about 19 days. All the edited DP2855H lines showed same heading date and maturity date.

TABLE 22
Flowering trait analysis of OsCMP1 gene edited (DP2855)
ZH11 rice plants at T2 generation in Beijing

| Days to | |
|---|---|
| 50% heading | Diff of heading date |

| Line ID | date (d) | ZH11-WT | DP2855N |
|---|---|---|---|
| DP2855H (Construct) | 90.1 | −18.2 | −18.2 |
| ZH11-WT | 108.3 | | |
| DP2855N | 108.3 | | |
| DP2855H.02B | 90.0 | −18.3 | −18.3 |
| DP2855H.05B | 90.0 | −18.3 | −18.3 |
| DP2855H.06B | 90.5 | −17.8 | −17.8 |
| DP2855H.07B | 90.0 | −18.3 | −18.3 |
| DP2855H.08B | 91.5 | −16.8 | −16.8 |
| DP2855H.09B | 89.0 | −19.3 | −19.3 |
| DP2855H.10B | 92.0 | −16.3 | −16.3 |
| DP2855H.11B | 89.5 | −18.8 | −18.8 |
| DP2855H.12B | 89.0 | −19.3 | −19.3 |
| DP2855H.14B | 89.0 | −19.3 | −19.3 |

### T1 Generation DP2925-HN

Nine T1 generation OsCMP1 modified rice lines (DP2925) were planted, and the ZH11-TC and the genome edited negative rice (DP2925N) plants which went through the transformation process and have the wild-type (un-mutated) OsCMP1 gene were used as controls. These nine modified lines has five genome editing patterns. The genome editing in the target sites in DP2925H.05B, DP2925H.06B and DP2925H.15B plants belong to mutation type 1; the genome editing in the target sites in DP2925H.12B plants belong to mutation type 2; the genome editing in the target sites in DP2925H.11B plants belong to mutation type 4; the genome editing in the target sites in DP2925H.02B, DP2925H.13B and DP2925H.14B plants belong to mutation type 5; and the genome editing in the target sites in DP2925H.17B plant belong to mutation type 8 in FIG. 6. The mutations resulted in translation shift, thereby resulting in early stops of the ORF. The predicted translated polypeptide has 104 or 249 to 263 amino acids. These plants were sowed in November and transplanted in the paddy field in December. The days from sowing to 50% heading date for the ZH11-TC control was 71 days, and for the DP2925N was 68 days. DP2925H rice plants exhibited earlier heading date than ZH11-TC and DP2925N rice plants at the construct level. As shown in Table 23, all the DP2925H rice lines exhibited an earlier heading date than ZH11-TC and DP2855N rice plants, and part of the genome edited rice plants exhibited early maturity date than the controls.

TABLE 23
Flowering trait analysis of OsCMP1 gene edited
(DP2925) rice plants at T1 generation in Hainan

| | Days to | | | Days to | | |
|---|---|---|---|---|---|---|
| | 50% heading | Diff of heading date | | maturity | Diff of maturity date | |
| Line ID | date (d) | ZH11-TC | DP2925N | date (d) | ZH11-TC | DP2925N |
| DP2925H | 61.6 | −8.9 | −6.1 | 108.7 | −1.3 | 1.0 |
| (Construct) | | | | | | |
| ZH11-TC | 70.5 | | | | | |
| DP2925N | 67.7 | | | | | |
| DP2925H.02B | 61 | −9.5 | −6.7 | 107 | −3.0 | −2.7 |
| DP2925H.05B | 62 | −8.5 | −5.7 | 106 | −4.0 | −3.7 |
| DP2925H.06B | 59 | −11.5 | −8.7 | 105 | −5.0 | −4.7 |
| DP2925H.11B | 61 | −9.5 | −6.7 | 113 | 3.0 | 3.3 |
| DP2925H.12B | 62 | −8.5 | −5.7 | 111 | 1.0 | 1.3 |
| DP2925H.13B | 61 | −9.5 | −6.7 | 109 | −1.0 | −0.7 |
| DP2925H.14B | 63 | −7.5 | −4.7 | 109 | −1.0 | −0.7 |
| DP2925H.15B | 63 | −7.5 | −4.7 | 108 | −2.0 | −1.7 |
| DP2925H.17B | 62 | −8.5 | −5.7 | 110 | 0.0 | 0.3 |

### T1 Generation DP2925-CS

Two homologous T1 generation OsCMP1 modified rice lines (DP2925) were planted in a Changsha field, and the ZH11-WT and DP2925N rice plants were used as controls. One nucleotide “T” inserted in the second target site and no mutation in the first target site in these two OsCMP1 modified rice plants, and resulted in translation shift, thereby resulting in early stop of the ORF. The mutation type belongs to type 5 in FIG. 6, and the predicted translated polypeptide has 263 amino acids. These plants were sowed in May and transplanted in the paddy field in June. Twenty plants from each line were planted in two rows. The days from sowing to 50% heading date for the ZH11-WT control and DP2925N were 76 days. These two DP2925H rice lines exhibited about 8 days earlier heading date than ZH11-WT and DP2925N rice plants and exhibited an early maturity date than the controls (Table 24).

TABLE 24
Flowering trait analysis of OsCMP1 gene edited (DP2925)
ZH11 rice plants at T1 generation in Changsha

| | Days to | | | Days to | | |
|---|---|---|---|---|---|---|
| | 50% heading | Diff of heading date | | maturity | Diff of maturity date | |
| Line ID | date (d) | ZH11-WT | DP2925N | date (d) | ZH11-WT | DP2925N |
| ZH11-WT | 76.0 | | | 121.0 | | |
| DP2925N | 76.0 | | | 121.0 | | |
| DP2925H.13B | 68.0 | −8.0 | −8.0 | 113.0 | −8.0 | −8.0 |
| DP2925H.14B | 68.0 | −8.0 | −8.0 | 113.0 | −8.0 | −8.0 |

### T1 Generation DP2925-BJ

The same two homologous T1 generation OsCMP1 modified rice lines (DP2925) were planted in a Beijing field, and the ZH11-WT and DP2925.N rice plants were used as controls. These plants were sowed in May and transplanted in the paddy field in June. Twenty plants from each line were planted in two rows. As shown in Table 25, the days from sowing to 50% heading date for the ZH11-WT control and DP2925N were 92 days. These two DP2925H rice lines exhibited about 20 days earlier heading date than ZH11-WT and DP2925N rice plants and exhibited an early maturity date than the controls.

TABLE 25
Flowering trait analysis of OsCMP1 gene edited (DP2925)
ZH11 rice plants at T1 generation in Beijing

| | Days to | | | Days to | | |
|---|---|---|---|---|---|---|
| | 50% heading | Diff of heading date | | maturity | Diff of maturity date | |
| Line ID | date (d) | ZH11-WT | DP2925N | date (d) | ZH11-WT | DP2925N |
| ZH11-WT | 91.8 | | | 139.0 | | |
| DP2925N | 92.5 | | | 139.0 | | |
| DP2925H.13B | 72.0 | −19.8 | −20.5 | 122.0 | −17.0 | −17.0 |
| DP2925H.14B | 71.0 | −20.8 | −21.5 | 122.0 | −17.0 | −17.0 |

### T2 Generation DP2925-CS

Ten homologous T2 OsCMP1 modified rice lines (DP2925) were planted, and the ZH11-WT and the genome edited negative rice (DP2925N) plants which went through the transformation process and have the wild-type (un-mutated) OsCMP1 gene were used as controls. These ten modified lines have five genome editing patterns. The genome editing in the target sites in DP2925H.02B and DP2925H.13B plants belong to mutation type 5; the genome editing in the target sites in other rice plants belong to mutation types 1 to 4 in FIG. 6. The mutations resulted in translation shift, thereby resulting in early stops of the ORF. The predicted translated polypeptide has 263 or 104 amino acids. These plants were sowed in May and transplanted in the paddy field in June. The days from sowing to 50% heading date for the ZH11-WT control and DP2925N rice plants were 76 days. DP2925H rice plants exhibited earlier heading date than ZH11-WT and DP2925N rice plants at the construct level. As shown in 26, all the genome edited DP2925H rice lines exhibited an earlier heading date than ZH11-WT and DP2855N rice plants and exhibited an early maturity date than the controls.

TABLE 26
Flowering trait analysis of OsCMP1 gene edited (DP2925)
ZH11 rice plants at T2 generation in Changsha

| | Days to | | | Days to | | |
|---|---|---|---|---|---|---|
| | 50% heading | Diff of heading date | | maturity | Diff of maturity date | |
| Line ID | date (d) | ZH11-WT | DP2925N | date (d) | ZH11-WT | DP2925N |
| DP2925H | 67.4 | −8.6 | −8.6 | 112.4 | −8.6 | −8.6 |
| (Construct) | | | | | | |
| ZH11-WT | 76.0 | | | 121.0 | | |
| DP2855N | 76.0 | | | 121.0 | | |
| DP2925H.01B | 67.0 | −9.0 | −9.0 | 112.0 | −9.0 | −9.0 |
| DP2925H.02B | 68.5 | −7.5 | −7.5 | 113.5 | −7.5 | −7.5 |
| DP2925H.03B | 68.5 | −7.5 | −7.5 | 113.5 | −7.5 | −7.5 |
| DP2925H.04B | 68.0 | −8.0 | −8.0 | 113.0 | −8.0 | −8.0 |
| DP2925H.06B | 67.0 | −9.0 | −9.0 | 112.0 | −9.0 | −9.0 |
| DP2925H.07B | 67.0 | −9.0 | −9.0 | 112.0 | −9.0 | −9.0 |
| DP2925H.09B | 66.0 | −10.0 | −10.0 | 111.0 | −10.0 | −10.0 |
| DP2925H.10B | 67.5 | −8.5 | −8.5 | 112.5 | −8.5 | −8.5 |
| DP2925H.12B | 66.5 | −9.5 | −9.5 | 111.5 | −9.5 | −9.5 |
| DP2925H.13B | 68.0 | −8.0 | −8.0 | 113.0 | −8.0 | −8.0 |

### T2 Generation DP2925-BJ

The same ten homologous T2 OsCMP1 modified rice lines (DP2925) were planted, and the ZH11-WT and DP2925N rice plants were used as controls. These plants were sowed in May and transplanted in the paddy field in June. The days from sowing to 50% heading date for the ZH11-WT control and DP2925N rice plants were 108 days. DP2925H rice plants exhibited earlier heading date than ZH11-WT and DP2925N rice plants at the construct level. As shown in Table 27, all the genome edited DP2925H rice lines exhibited an earlier heading date than ZH11-WT and DP2855N rice plants and exhibited an early maturity date than the controls.

TABLE 27
Flowering trait analysis of OsCMP1 gene edited (DP2925)
ZH11 rice plants at T2 generation in Beijing

| Days to | |
|---|---|
| 50% heading | Diff of heading date |

| Line ID | date (d) | ZH11-WT | DP2925N |
|---|---|---|---|
| DP2925H (Construct) | 89.9 | −18.4 | −17.6 |
| ZH11-WT | 108.3 | | |
| DP2855N | 107.5 | | |
| DP2925H.01B | 88.5 | −19.8 | −19 |
| DP2925H.02B | 92.0 | −16.3 | −15.5 |
| DP2925H.03B | 91.5 | −16.8 | −16 |
| DP2925H.04B | 90.0 | −18.3 | −17.5 |
| DP2925H.06B | 90.0 | −18.3 | −17.5 |
| DP2925H.07B | 89.0 | −19.3 | −18.5 |
| DP2925H.09B | 87.7 | −20.6 | −19.8 |
| DP2925H.10B | 87.0 | −21.3 | −20.5 |
| DP2925H.12B | 89.5 | −18.8 | −18.0 |
| DP2925H.13B | 92.0 | −16.3 | −15.5 |

The experiments in Hainan (18 °30′N), Changsha (28 °11′N) and Beijing (40 °13′N) field demonstrated that the edited DP2855 and DP2925 rice plants can head earlier and that the higher the latitude the difference of heading date is greater. These results demonstrate that genome editing of the OsCMP1 gene, even if one nucleotide insertion, can regulate the flowering time trait in rice.

### 2) Daohuaxiang 2

### T1 Generation RL2855-BJ

OsCMP1 genome edited Daohuaxiang 2 seedlings (RL2855) were planted in a Beijing field (40 °13′N), and the wild type Daohuaxiang 2 and the genome edited negative rice (RL2855N) plants which went through the transformation process and have the wild-type (un-mutated) OsCMP1 gene were used as controls. The genome editing in the target sites in the tested rice plants belong to mutation type 2 in FIG. 5. The mutations resulted in translation shift, thereby resulting in early stop of the ORF. The predicted translated polypeptide has 104 amino acids. The rice plants were transplanted in paddy field in June. The phenotype was recorded during plant growth and the first heading date is used. As shown in Table 28, the average days to first heading date of RL2855 rice plants were less by about 25 days than that of wild type Daohuaxiang 2 and RL2855N rice plants. These results indicate that Genome edited RL2855 rice plants headed earlier than the controls.

TABLE 28
Flowering trait of OsCMP1 gene edited (RL2855) Daohuaxiang
2 rice plants at T1 generation in Beijing

| | | Average days | Diff | Diff |
|---|---|---|---|---|
| | Plants | to first heading | compared | compared |
| Line ID | number | date (d) | with WT | with RL2855N |
| RL2855 | 66 | 59.9 ± 5.1 | −25.4 | −25.9 |
| (Construct) | | | | |
| Daohuaxiang | 30 | 85.3 ± 2.8 | | |
| RL2855N | 12 | 85.8 ± 1.5 | | |
| RL2855H.09B | 8 | 60.3 ± 0.7 | −25.0 | −25.5 |
| RL2855H.11B | 11 | 60.7 ± 4.0 | −24.5 | −25.1 |
| RL2855H.13B | 8 | 60.1 ± 1.0 | −25.1 | −25.7 |
| RL2855P.24B | 11 | 61.8 ± 4.2 | −23.4 | −24.0 |
| RL2855P.34B | 11 | 54.9 ± 4.1 | −30.4 | −30.9 |
| RL2855P.38B | 9 | 56.0 ± 4.6 | −29.3 | −29.8 |
| RL2855P.43B | 8 | 66.6 ± 5.0 | −18.6 | −19.2 |

### T1 Generation RL2855-Heilongjiang

OsCMP1 genome edited Daohuaxiang 2 seedlings (RL2855) were planted in a Heilongjiang field (45 °53′N), and the wild type Daohuaxiang 2 and RL2855N rice plants were used as controls. The rice plants were transplanted in paddy field in May. The phenotype was recorded during plant growth and the first heading date is used. As shown in Table 29, the average days to first heading date of RL2855 rice plants were less by about 14 days than that of wild type Daohuaxiang 2 rice plants, and all the edited rice plants showed an earlier heading date. These results indicate that Genome edited RL2855 rice plants headed earlier than the controls in Heilongjiang, 45 °53′N.

TABLE 29
Flowering trait of OsCMP1 gene edited (RL2855) Daohuaxiang
2 rice plants at T1 generation in Heilongjiang

| | | Average days | Diff | Diff |
|---|---|---|---|---|
| | Plants | to first heading | compared | compared |
| Line ID | number | date (d) | with WT | with RL2855N |
| RL2855 | 100 | 110.1 ± 3.0 | −13.7 | −15.9 |
| (Construct) | | | | |
| Daohuaxiang | 16 | 123.8 ± 2.2 | 0.0 | −2.2 |
| RL2855N | 10 | 125.4 ± 0.5 | 1.6 | −0.6 |
| RL2855H.01 | 32 | 110.6 ± 2.6 | −13.2 | −15.4 |
| RL2855H.02 | 16 | 109.3 ± 3.0 | −14.6 | −16.7 |
| RL2855P.01 | 16 | 110.9 ± 2.3 | −12.9 | −15.1 |
| RL2855P.02 | 10 | 111,2 ± 3.4 | −12.6 | −14.8 |
| RL2855P.09 | 2 | 109.0 ± 0.0 | −14.8 | −17.0 |
| RL2855P.42 | 10 | 110.4 ± 3.6 | −13.4 | −15.6 |
| RL2855P.44 | 4 | 110.5 ± 1.7 | −13.3 | −15.5 |
| RL2855P.45 | 2 | 109.0 ± 0.0 | −14.8 | −17.0 |
| RL2855P.57 | 8 | 106.5 ± 3.4 | −17.3 | −19.5 |

### T1 Generation RL2925-BJ

OsCMP1 genome edited Daohuaxiang 2 seedlings (RL2925) were planted in a Beijing field (40 °13′N), and the wild type Daohuaxiang 2 and the genome edited negative rice (RL2925N) plants which went through the transformation process and have the wild-type (un-mutated) OsCMP1 gene were used as controls. The genome editing in the target sites in the tested rice plants belong to mutation type 1 to 4 in FIG. 6. The mutations resulted in translation shift, thereby resulting in early stop of the ORF. The predicted translated polypeptide has 104 amino acids. The rice plants were transplanted in paddy field in June. The phenotype was recorded during plant growth and the first heading date is used. As shown in Table 30, the average days to first heading date of RL2925 rice plants were less by about 27 days than that of wild type Daohuaxiang 2 rice plants, and all the edited rice lines showed earlier heading date. These results indicate that Genome edited RL2925 rice plants headed earlier than the controls in Beijing.

TABLE 30
Flowering trait of OsCMP1 gene edited (RL2925) Daohuaxiang
2 rice plants at T1 generation in Beijing

| | | Average days | Diff | Diff |
|---|---|---|---|---|
| | Plants | to first heading | compared | compared |
| Line ID | number | date (d) | with WT | with RL2925N |
| RL2925 | 70 | 58.3 ± 7.1 | −27.0 | −28.4 |
| (Construct) | | | | |
| Daohuaxiang | 30 | 85.3 ± 2.8 | | |
| RL2925N | 20 | 86.7 ± 1.9 | | |
| RL2925H.01B | 9 | 55.7 ± 6.3 | −29.6 | −31.0 |
| RL2925H.02B | 12 | 54.8 ± 3.1 | −30.4 | −31.9 |
| RL2925H.03B | 9 | 68.4 ± 7.7 | −16.8 | −18.3 |
| RL2925H.04B | 9 | 59.4 ± 6.5 | −25.8 | −27.3 |
| RL2925P.25B | 12 | 59.4 ± 8.4 | −25.9 | −27.3 |
| RL2925P.42B | 12 | 54.6 ± 1.6 | −30.7 | −32.1 |
| RL2925P.44B | 7 | 57.7 ± 4.6 | −27.6 | −29.0 |

### T1 Generation RL2925-Heilongjiang

OsCMP1 genome edited Daohuaxiang 2 seedlings (RL2925) were planted in Heilongjiang field (45 °53′N), and the wild type Daohuaxiang 2 rice plants were used as controls. The rice plants were transplanted in paddy field in May. The phenotype was recorded during the plant growth and the first heading date is used. As shown in Table 31, the average days to first heading date of RL2925 rice plants were less by about 15 days than that of wild type Daohuaxiang rice plants, and all the edited rice plants showed earlier heading date. These results indicate that Genome edited RL2925 rice plants headed earlier than the controls in Heilongjiang, 45 °53′N.

TABLE 31
Flowering trait of OsCMP1 gene edited (RL2925) Daohuaxiang
2 rice plants at T1 generation in Heilongjiang

| | | Average days | Diff |
|---|---|---|---|
| | Plants | to first heading | compared |
| Line ID | number | date (d) | with WT |
| RL2925 | 156 | 109 ± 4.7 | −14.9 |
| Daohuaxiang | 16 | 123.8 ± 2.2 | 0.0 |
| RL2925H.01 | 22 | 110.6 ± 2.8 | −13.2 |
| RL2925H.02 | 30 | 108.5 ± 6.1 | −15.3 |
| RL2925H.03 | 30 | 111,5 ± 2.2 | −12.3 |
| RL2925H.04 | 26 | 110.8 ± 3.7 | −13.0 |
| RL2925P.25 | 20 | 104.7 ± 5.3 | −19.1 |
| RL2925P.42 | 28 | 106.8 ± 3.8 | −17.0 |

The experiments in Beijing (40 °13′N) and Heilongjiang (45 °53′N) field demonstrated that the edited RL2855 and RL2925 Daohuaxiang 2 rice plants can head earlier and that the higher the latitude the difference in heading date is smaller. These results demonstrate that genome editing the OsCMP1 gene, even if one nucleotide insertion, can regulate the flowering time trait.

The above experiments demonstrated that genome editing in OsCMP1 gene affects the flowering time trait, and that one nucleotide insertion, two nucleotide deletions, or small fragment deletion in OsCMP1 gene promote earlier flowering in different varieties in different latitude, such as Hainan (18 °30′N), Changsha (28 °11′N), Beijing (40 °13′N) and Heilongjiang (45 °53′N).

1-6. (canceled)

7. A modified plant having increased expression of a flowering time-regulating polynucleotide sequence encoding a polypeptide comprising an amino acid sequence that is at least 90% identical to SEQ ID NO: 7, wherein the plant exhibits delayed flowering time when compared to a control plant not having said increased expression under the same conditions.

8. The plant of claim 7, wherein the plant comprises a recombinant DNA construct comprising the flowering time-regulating polynucleotide operably linked to at least one regulatory element.

9. The plant of claim 7, wherein the plant comprises a modification of a regulatory element operably linked to the flowering time-regulating polynucleotide thereby increasing the expression of an endogenous polynucleotide.

10. A modified plant having reduced expression or function of a flowering time-regulating polynucleotide sequence encoding a polypeptide comprising an amino acid sequence that is at least 90% identical to SEQ ID NO: 7, wherein the plant exhibits earlier flowering time when compared to a control plant planted under the same conditions.

11. The plant of claim 10, wherein the plant comprises a suppression DNA construct, wherein the suppression DNA construct comprises a suppression element operably linked to at least one regulatory element, wherein the suppression element comprises at least 100 contiguous base pairs of (a) a polynucleotide with nucleotide sequence of at least 85% identity to SEQ ID NO: 5 or 6; (b) a polynucleotide encoding a polypeptide with amino acid sequence of at least 90% identity to SEQ ID NO: 7; or (c) the full complement of the nucleotide sequence of (a) or (b).

12. The plant of claim 10, wherein the plant comprises a mutated genomic locus in a flowering time-regulating gene comprising the polynucleotide sequence or its promoter by (a) introducing a DNA fragment or deleting a DNA fragment or replacing a DNA fragment or introducing (b) one or more nucleotide changes in the genomic region comprising the endogenous flowering time-regulating gene and its promoter.

13. The plant of claim 12, wherein the plant comprises a mutated genomic locus in the flowering time-regulating gene comprising the polynucleotide, wherein the function of the flowering time-regulating gene is reduced in the plant, when compared to the control plant.

14. The plant of claim 13, wherein the plant comprises a mutated promoter of the flowering time-regulating gene, wherein the expression of the flowering time-regulating polypeptide is decreased in the plant, when compared to a control plant.

15. The plant of claim 7, wherein the said plant is selected from the group consisting of rice, maize, soybean, sunflower, sorghum, canola, wheat, alfalfa, cotton, barley, millet, sugar cane and switchgrass.

16-21. (canceled)

22. A method of regulating flowering time in plant comprising altering the expression or function of a polynucleotide sequence encoding a polypeptide comprising an amino acid sequence that is at least 90% identical to SEQ ID NO: 7 in a plant.

23. (canceled)

24. The method of claim 22, wherein the expression or the function of the polynucleotide is altered by a step selected from the group consisting of:
(a) increasing the expression of the polynucleotide by expressing in the plant a recombinant DNA construct comprising the polynucleotide operably linked to at least one regulatory element;
(b) increasing the expression of the polynucleotide by introducing a targeted mutation in a regulatory element at a genomic locus comprising the polynucleotide;
(c) reducing the function of an endogenous polynucleotide encoding the polypeptide having an amino acid sequence of at least 90% sequence identity compared to SEQ ID NO: 7; and
(d) decreasing the expression of the polynucleotide by introducing into the plant a suppression DNA construct, wherein the suppression DNA construct comprises a suppression element that reduces the expression of an endogenous polynucleotide encoding the polypeptide having an amino acid sequence of at least 90% identity to SEQ ID NO: 7.

25. The method of claim 24, wherein the increased expression of the polynucleotide in the plant delays flowering time when compared to a control plant not having said increased expression.

26. The method of claim 24, wherein reducing the expression of the polynucleotide in the plant promotes earlier flowering time when compared to a control plant not having said reduced expression.

27. The method of claim 24, wherein reducing the function of the polynucleotide in the plant promotes earlier flowering time when compared to a control plant.

28. The method of claim 27, wherein the function of the polynucleotide is reduced by targeting the nucleotide sequence of SEQ ID NO:5 or 6 using an sgRNA comprising a nucleotide sequence of at least one of SEQ ID NOs: 18-39.

29. The method of claim 28, wherein the sgRNA is nucleotide sequence of SEQ ID NO: 22.

30. The method of claim 28, wherein the sgRNA is nucleotide sequence of SEQ ID NO: 22 and 18.

31-32. (canceled)

33. The method of claim 24, wherein the targeted mutation is introduced using zinc finger nuclease, Transcription Activator-Like Effector Nuclease (TALEN), CRISPR-cas, guided Cas endonuclease, meganuclease or CRISPR-Cas ribonucleoprotein complexes.

34-37. (canceled)

38. The plant of claim 10, wherein the said plant is selected from the group consisting of rice, maize, soybean, sunflower, sorghum, canola, wheat, alfalfa, cotton, barley, millet, sugar cane and switchgrass.

39. The method of claim 24, wherein the regulatory element comprises the nucleotide sequence of SEQ ID NO: 8.


SEQUENCE LISTING (69 sequences)

SEQ ID NO: 1 - DNA, 226 bp, Oryza sativa
attaatccataattaacatatgtgatgctatagtaaacatgttctaatcatagattaattaggcttaaaaaatttgtctcgcgaattagctcttatttaatcaattagttttattattattctacgttcaatagttctaactagtgtccaaacatccgaagtgacaaagactaaaatttagtccttagatccaaacaccacctaagacaggaatctagatatctag

SEQ ID NO: 2 - DNA, 677 bp, Oryza sativa
gccccatttcctttgcagttccatttacagtccgcctagaaataaaagggtccggtatccaagaaaatctttcctgtagtagtgaaactgctttaaatttgaccaagtttataaaaaaagtagtaacattttcaacccaagataaacttattataaaaatatatttaattattgatttgataaaactaatttagtattataaatattactatatttgtctataaacttagtcaatcttgaaacaatatgactttgatcaaagtcaaaacgtcttataatctgaaacggaggaagtagatagataagatacttgaattgatcatgtgcgcacgttatatgcaaaatcaaggcttaataattaagatcattgatccgatttcatgagtgcatacatactaattatttagagtttactatgtacaaagtcgattccacttatctgaaaataaatctacctcgctataaaggttacgtaaacatatcgcagtcatattagtataccgtggtttgagaacccacaaaagatatctacatgtcatatcacacatataaatctgtattactcccttcgtcacataatataaaggattttaaaagtatatggcacatcatagtttgacgaatctagataaggagcctgtctagacctccctaaaaaatcccttatattataggac

SEQ ID NO: 3 - DNA, 910 bp, Oryza sativa
cctcaatctattatcagtcatatgtatgagagcaaccacggtatatcgaaaggtaaccttatggcaatgattacatttataaagtggaacacatacattgtgagaaatagctttagcttagagtgcatcttatacttaaggtctaccacaagcataaagaaaatattctctctctcatttatgtcatgaacaagagctgaaaagcaggtttttctatctcataagggccccatattattgttaaatttcagcaagggattagctaaaaaactgttgcaggttttttgagtaaaaaactttcaaatctaactatagtacataattttactagaactacactataataattatataacttgtatagatgtattaaaataatatatgcaactttatatctaatttgatagagataataatgtagttactgtaactagggtataactggagtataagtaacatgtaacttgctaattttttaaaaaacttgcaagctggtgggatcgaggtcctgggttcgaaccccatgcagcgcacaaattatgtttctcacacgggatttttttccatgaacgcgccagcacgaatcttgagatgaatctgacggtcaaaaattcgaaagaatttacccccgttttctgtcgatagaaaactagcaaatccgtttcagcaatagcattatgggaattgctttaacaacaatcaaactattcatgggccacttctaagatcacactagacccatatacattgagattgccctgatatatccatctaattcatggacattttcctagtcttggggggatataatataataggagctagaggggggcatgggtagtgaagtccagccagcgcagaagatccttggggggatctggttgcaatggggatggccaatgaggagtcg

SEQ ID NO: 4 - DNA, 559 bp, Oryza sativa
gtcatcgaacagccaggccacgctctcgccgtcgtcgccgccgccgtcgttgccgaagaactggaactcgtgcaccggtggcgccggggcgccgatcccctggcacgcactcggcgggaagacgaaggggaatccatcatcatcgtggcgatggcgggagcaacagccgccaccgtcggcgccgcacaggccacatccttctccggctgctggtcccatcgacatgaacggataaatcaaactcgatcgagctacaaacaacaactagctagagctagctagtaagttagctatagcaggtgaggtcacttgagctcgatcagctcagctagctagggttgtgaaatgcttgtgatcactgatcgatcagcgagatcgatgacttgtaataatataggtacaagaagaagaagaagaagaagacagggcaagttggggatgagggaaaggtaggggggccattgcgagatcatattatgatcgatcgaggagagcggggcgatctgtggatggatttggtccttacgtggaggaatccggccgccttttttcacctgat

SEQ ID NO: 5 - DNA, 2786 bp, Oryza sativa
atggggatggccaatgaggagtcgccaaattatcaggtgaaaaaaggcggccggattcctccacgtaaggaccaaatccatccacagatcgccccgctctcctcgatcgatcataatatgatctcgcaatggcccccctacctttccctcatccccaacttgccctgtcttcttcttcttcttcttcttgtacctatattattacaagtcatcgatctcgctgatcgatcagtgatcacaagcatttcacaaccctagctagctgagctgatcgagctcaagtgacctcacctgctatagctaacttactagctagctctagctagttgttgtttgtagctcgatcgagtttgatttatccgttcatgtcgatgggaccagcagccggagaaggatgtggcctgtgcggcgccgacggtggcggctgttgctcccgccatcgccacgatgatgatggattccccttcgtcttcccgccgagtgcgtgccaggggatcggcgccccggcgccaccggtgcacgagttccagttcttcggcaacgacggcggcggcgacgacggcgagagcgtggcctggctgttcgatgactacccgccgccgtcgcccgttgctgccgccgccgggatgcatcatcggcagccgccgtacgacggcgtcgtggcgccgccgtcgctgttcaggaggaacaccggcgccggcgggctcacgttcgacgtctccctcggcgaacggcccgacctggacgccgggctcggcctcggcggcggcggcggccggcacgccgaggccgcggccagcgccaccatcgtgagtatcaatccaataatcctgatccggccggcatgatcggctcgattgagccgtgtcgattattaatttccatcttatatattattaattgatgaattcttgattgattcatcgatcctcctcgtcttttcttggcttctttgtttttgttatttagtcaaaaacaactcttcatttctgctgcctatatgccgtacaacttcaaactatcaaaggtcaaataatcgatcaatatataccaagtttgaattaatttggagcttaattaattaattactggcttgcagcagctggtttatagtattgtttctagctatatatgtgagggccgtgtgtgggatgtgatttgcatctttcgatgacgacttaattaattcgatgatatatttcattgcatatgcatacggatccagcctctgtctatactgtacgattccacatacgtatatgtacggttaagtcagtatatatacttttagatagtcgcgtgtgcttttcgagttcggtagctatattttagattgtaaaaacaagtcagaggctaattttataatctagaaatacttatttccccatatataagcgtatgttaaatattgatggtgtaatctacttatatgtcaggaaacatcattgcttgctttctggcgctttcttctacatatcagtagaggaaaatggaaaaaaaaagatgaattttgatgttgtagtttgctatattcagcatatataccatcagttatacatatgcagatcttgctaaaaccaaaataaaaatagaactgtaaggagatattgtgcttctcggtctatttacttacagtttgttgagaagtaatacgagcaagcaaatgtatatatatatttctttagaactgcaaggagatgcatatacatgtgtgattcaaacacacgtactgcacattcaaactataaaaacaacttgattgccgtagaagttaaaagggagacatatccatgggtttcggattctaaatcaatctatgtgtaaatgaaactttagtatagtaggaaataggttttcaaaaaaaaaagtatagtaggaaatagtatgtgtatatgcctttttaacccttaattacaagttgttataattcagtgttaacaaagtcacggactcacagagtgtgcccttacacaatttcagactaatttgtaaatgcatcgatcgtcacattttatgtggttcaattatctgacacagttaattaatggtggccgatcgatgtatgctcttctagctttccagctatatgcgtatgtaataaatgaataaaacgtgtaggatgaaatgtgaatacgcatcattgtaattaatttgattaatgctagtaaaaaatctgcaaatttgtctttttgaaattaaaatatgccttataaaattaatggacccaggcccctagggcaaaatatattggggcacaaaatcatgtccatatatacattcttatttgaaagtagactctgaaacaaaatatgcccatataaatcaagggaggttacaactaactgcatttgcttatgcgtacatctggattgtaacttctatgttttgtacatacgatgattaattgtattcgagcttcttaattgtacatctattaactaactagttttgcagatgtcatattgtgggagcacgttcactgacgcagcgagctcgatgcccaaggagatggtggccgccatggccgatgatggggagagcttgaacccaaacacggtggttggcgcaatggtggagagggaggccaagctgatgaggtacaaggagaagaggaagaagaggtgctacgagaagcaaatccggtacgcgtccagaaaagcctatgccgagatgaggccccgagtgagaggtcgcttcgccaaagaacctgatcaggaagctgtcgcaccgccatccacctatgtcgatcctagtaggcttgagcttggacaatggttcagatagat

SEQ ID NO: 6 - DNA, 864 bp, Oryza sativa
atggggatggccaatgaggagtcgccaaattatcaggtgaaaaaaggcggccggattcctccacctcgatcgagtttgatttatccgttcatgtcgatgggaccagcagccggagaaggatgtggcctgtgcggcgccgacggtggcggctgttgctcccgccatcgccacgatgatgatggattccccttcgtcttcccgccgagtgcgtgccaggggatcggcgccccggcgccaccggtgcacgagttccagttcttcggcaacgacggcggcggcgacgacggcgagagcgtggcctggctgttcgatgactacccgccgccgtcgcccgttgctgccgccgccgggatgcatcatcggcagccgccgtacgacggcgtcgtggcgccgccgtcgctgttcaggaggaacaccggcgccggcgggctcacgttcgacgtctccctcggcgaacggcccgacctggacgccgggctcggcctcggcggcggcggcggccggcacgccgaggccgcggccagcgccaccatcatgtcatattgtgggagcacgttcactgacgcagcgagctcgatgcccaaggagatggtggccgccatggccgatgatggggagagcttgaacccaaacacggtggttggcgcaatggtggagagggaggccaagctgatgaggtacaaggagaagaggaagaagaggtgctacgagaagcaaatccggtacgcgtccagaaaagcctatgccgagatgaggccccgagtgagaggtcgcttcgccaaagaacctgatcaggaagctgtcgcaccgccatccacctatgtcgatcctagtaggcttgagcttggacaatggttcagatag

SEQ ID NO: 7 - protein, 287 aa, Oryza sativa
MGMANEESPNYQVKKGGRIPPPRSSLIYPFMSMGPAAGEGCGLCGADGGGCCSRHRHDDDGFPFVFPPSACQGIGAPAPPVHEFQFFGNDGGGDDGESVAWLFDDYPPPSPVAAAAGMHHRQPPYDGVVAPPSLFRRNTGAGGLTFDVSLGERPDLDAGLGLGGGGGRHAEAAASATIMSYCGSTFTDAASSMPKEMVAAMADDGESLNPNTVVGAMVEREAKLMRYKEKRKKRCYEKQIRYASRKAYAEMRPRVRGRFAKEPDQEAVAPPSTYVDPSRLELGQWFR

SEQ ID NO: 8 - DNA, 4000 bp, Oryza sativa
tatcctaagataggaatgccctgtttggtttagggactaaattttaatccctatcacatcgaatgtttgacactaattagaagtactaaacgtagactaatgccaaaacccattccataaccttggactaatttgcgagataaatctattaagcctaattaatccataattaacatatgtgatgctatagtaaacatgttctaatcatagattaattaggcttaaaaaatttgtctcgcgaattagctcttatttaatcaattagttttattattattctacgttcaatagttctaactagtgtccaaacatccgaagtgacaaagactaaaatttagtccttagatccaaacaccacctaagacaggaatctagatatctagattcgttgttctatgataggccacgtcatatcctagattatttctttttggaacggagggagtaagtatactatcgatcataatggcgacgtgaccaataatatacgtgtacaattaccaactgtgtactccctctgtcctataatataagggattttttagggaggtctagacaggctccttatctagattcgtcaaactatgatgtgccatatacttttaaaatcctttatattatgtgacgaagggagtaatacagatttatatgtgtgatatgacatgtagatatcttttgtgggttctcaaaccacggtatactaatatgactgcgatatgtttacgtaacctttatagcgaggtagatttattttcagataagtggaatcgactttgtacatagtaaactctaaataattagtatgtatgcactcatgaaatcggatcaatgatcttaattattaagccttgattttgcatataacgtgcgcacatgatcaattcaagtatcttatctatctacttcctccgtttcagattataagacgttttgactttgatcaaagtcatattgtttcaagattgactaagtttatagacaaatatagtaatatttataatactaaattagttttatcaaatcaataattaaatatatttttataataagtttatcttgggttgaaaatgttactactttttttataaacttggtcaaatttaaagcagtttcactactacaggaaagattttcttggataccggacccttttatttctaggcggactgtaaatggaactgcaaaggaaatggggcggggggggggggggtacatgcgccagaccatcgcccgcataggaaaatcaattttcgtgtgcgggtcactatatagtccaccctacacgaaaatagtccaccctacatcgagcgctcgatatcctttcgcgcccctcgcccctccgcccactgccccactacctccctctctcccacctcactctctccctctctcccactccactcctctcctcccactcagcggagacagcaggcagcggtgggtgatatagcggcagcgcttggcccctccctccctctccctctgctgtgggtgggaagaggggcagtgacagcggtggcggcgggcgaccacggcggcgtgcggcccctcccctccctgcccttgcatatccggtggaggggaagcatgggggcctggtggatccggcagagctgcgagaggcgacgtgcggtggcgggaaggaggcagcccgcgcccctcggtggcgggaggcagttggacagagtcggcctgcgtccctcggcggtgttaggtggtgggacagaggcggcccgcgccggatctggaggtgtggcagtgtcagcggctggtgtggaggtgtgactatcggcaggaggtgcgacagtggcatcagcgatcgatgtgagttgatgcccctcccctcccctcccctccccttgcagatctagcggaggggaggagcgtcttcagctggcgactatggatccgacaccagcgacgggagcgggtggtagatctgtagccggcgaccgcgggggcaacggtggcgggcctcggctagggcagcggaggattaggattagggttttgtttttattttttttgtttcgatattttattatctcgtgcgggcagcttaagcgcccgcacgcgaaaatccgattttcgcgtgcgggtgaggcacctgcatgtgaaaatcacgattttcccagacccctgggtgcgagcgggccgaccatccgcgcagaaaaattattttcgaccgcacgcaaaaatctttattgtagtagtgtttgactttgactaaagtcaaaacgtcttataactgaaatggagggagtagaaaacaatattttccctttagtaaactcatgaaaccatccggctttgcaatttcaatagacactaaaatttctgttgttgccaaaattttgaaaatttcatgaatttcgatgttttctaaccggaaatattttaagttttaacatatttttgaccaaatttacaaatatttaaaaaaattagaaaaattttgggggagatttgtgcttgccggtggagcaaaatttgtttttttgaacgactcgcacaagacggtgcgaagttttattgatagagcagaaaaaaattacaagattaaccaagaaaaaggaaaaaatataccaccacccacacaccgacagcgccaacacatgggtccgggagaaggctagcaccggacaggccgtcactaagcgtgaccaaccgccactaggccaacaaaggaacacagaagagaacgcccaaaaaacgcccttacaaccaacacaaagcccaactccatctagattgtgcttgcaccaatcgttcgagtggtttcggcaaggggatgcctaaacgacgtctccaacgagagaagcgacggaaaaccgtcgccaccatccgtcagggactcaaaagagccaagattgggctttcgcctggcaaacacctttgagggatgagacggcacgacaacgccctcaggagggggaatgaaccatcgttgtcggtccagtcaagaccaagctgggttttcacccaccgctcatcacctgcaaatccacggctgacgcaccgatgctccaccacagctcaacctctgccgatgtgtgggaccactgcaccccgtccccgccggctagccttcgtgcgccgaagaccgcgccacaccctcaatctattatcagtcatatgtatgagagcaaccacggtatatcgaaaggtaaccttatggcaatgattacatttataaagtggaacacatacattgtgagaaatagctttagcttagagtgcatcttatacttaaggtctaccacaagcataaagaaaatattctctctctcatttatgtcatgaacaagagctgaaaagcaggtttttctatctcataagggccccatattattgttaaatttcagcaagggattagctaaaaaactgttgcaggttttttgagtaaaaaactttcaaatctaactatagtacataattttactagaactacactataataattatataacttgtatagatgtattaaaataatatatgcaactttatatctaatttgatagagataataatgtagttactgtaactagggtataactggagtataagtaacatgtaacttgctaattttttaaaaaacttgcaagctggtgggatcgaggtcctgggttcgaaccccatgcagcgcacaaattatgtttctcacacgggatttttttccatgaacgcgccagcacgaatcttgagatgaatctgacggtcaaaaattcgaaagaatttacccccgttttctgtcgatagaaaactagcaaatccgtttcagcaatagcattatgggaattgctttaacaacaatcaaactattcatgggccacttctaagatcacactagacccatatacattgagattgccctgatatatccatctaattcatggacattttcctagtcttggggggatataatataataggagctagaggggggcatgggtagtgaagtccagccagcgcagaagatccttggggggatctggttgca

SEQ ID NO: 9 - DNA, 21 bp, Artificial Sequence
  Forward primer for cloning gDNA of OsCMP1 gene
atggggatggccaatgaggag

SEQ ID NO: 10 - DNA, 26 bp, Artificial Sequence
  Reverse primer for cloning gDNA of OsCMP1 gene
atctatctgaaccattgtccaagctc

SEQ ID NO: 11 - DNA, 20 bp, Artificial Sequence
  Forward primer for real-time RT-PCR analysis of OsCMP1 gene
acgagaagcaaatccggtac

SEQ ID NO: 12 - DNA, 21 bp, Artificial Sequence
  Reverse primer for real-time RT-PCR analysis of OsCMP1 gene
tgatcaggttctttggcgaag

SEQ ID NO: 13 - DNA, 1934 bp, Zea mays
cagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctcttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgct

SEQ ID NO: 14 - DNA, 4206 bp, Artificial Sequence
  Nucleotide sequence of nuclear localization sequence and Cas9 gene
atggcccctaagaagaagagaaaggtcggtattcacggcgttcctgcggcgatggacaagaagtatagtattggtctggacattgggacgaattccgttggctgggccgtgatcaccgatgagtacaaggtcccttccaagaagtttaaggttctggggaacaccgatcggcacagcatcaagaagaatctcattggagccctcctgttcgactcaggcgagaccgccgaagcaacaaggctcaagagaaccgcaaggagacggtatacaagaaggaagaataggatctgctacctgcaggagattttcagcaacgaaatggcgaaggtggacgattcgttctttcatagattggaggagagtttcctcgtcgaggaagataagaagcacgagaggcatcctatctttggcaacattgtcgacgaggttgcctatcacgaaaagtaccccacaatctatcatctgcggaagaagcttgtggactcgactgataaggcggaccttagattgatctacctcgctctggcacacatgattaagttcaggggccattttctgatcgagggggatcttaacccggacaatagcgatgtggacaagttgttcatccagctcgtccaaacctacaatcagctctttgaggaaaacccaattaatgcttcaggcgtcgacgccaaggcgatcctgtctgcacgcctttcaaagtctcgccggcttgagaacttgatcgctcaactcccgggcgaaaagaagaacggcttgttcgggaatctcattgcactttcgttggggctcacaccaaacttcaagagtaattttgatctcgctgaggacgcaaagctgcagctttccaaggacacttatgacgatgacctggataaccttttggcccaaatcggcgatcagtacgcggacttgttcctcgccgcgaagaatttgtcggacgcgatcctcctgagtgatattctccgcgtgaacaccgagattacaaaggccccgctctcggcgagtatgatcaagcgctatgacgagcaccatcaggatctgacccttttgaaggctttggtccggcagcaactcccagagaagtacaaggaaatcttctttgatcaatccaagaacggctacgctggttatattgacggcggggcatcgcaggaggaattctacaagtttatcaagccaattctggagaagatggatggcacagaggaactcctggtgaagctcaatagggaggaccttttgcggaagcaaagaactttcgataacggcagcatccctcaccagattcatctcggggagctgcacgccatcctgagaaggcaggaagacttctacccctttcttaaggataaccgggagaagatcgaaaagattctgacgttcagaattccgtactatgtcggaccactcgcccggggtaattccagatttgcgtggatgaccagaaagagcgaggaaaccatcacaccttggaacttcgaggaagtggtcgataagggcgcttccgcacagagcttcattgagcgcatgacaaattttgacaagaacctgcctaatgagaaggtccttcccaagcattccctcctgtacgagtatttcactgtttataacgaactcacgaaggtgaagtatgtgaccgagggaatgcgcaagcccgccttcctgagcggcgagcaaaagaaggcgatcgtggaccttttgtttaagaccaatcggaaggtcacagttaagcagctcaaggaggactacttcaagaagattgaatgcttcgattccgttgagatcagcggcgtggaagacaggtttaacgcgtcactggggacttaccacgatctcctgaagatcattaaggataaggacttcttggacaacgaggaaaatgaggatatcctcgaagacattgtcctgactcttacgttgtttgaggatagggaaatgatcgaggaacgcttgaagacgtatgcccatctcttcgatgacaaggttatgaagcagctcaagagaagaagatacaccggatggggaaggctgtcccgcaagcttatcaatggcattagagacaagcaatcagggaagacaatccttgactttttgaagtctgatggcttcgcgaacaggaattttatgcagctgattcacgatgactcacttactttcaaggaggatatccagaaggctcaagtgtcgggacaaggtgacagtctgcacgagcatatcgccaaccttgcgggatctcctgcaatcaagaagggtattctgcagacagtcaaggttgtggatgagcttgtgaaggtcatgggacggcataagcccgagaacatcgttattgagatggccagagaaaatcagaccacacaaaagggtcagaagaactcgagggagcgcatgaagcgcatcgaggaaggcattaaggagctggggagtcagatccttaaggagcacccggtggaaaacacgcagttgcaaaatgagaagctctatctgtactatctgcaaaatggcagggatatgtatgtggaccaggagttggatattaaccgcctctcggattacgacgtcgatcatatcgttcctcagtccttccttaaggatgacagcattgacaataaggttctcaccaggtccgacaagaaccgcgggaagtccgataatgtgcccagcgaggaagtcgttaagaagatgaagaactactggaggcaacttttgaatgccaagttgatcacacagaggaagtttgataacctcactaaggccgagcgcggaggtctcagcgaactggacaaggcgggcttcattaagcggcaactggttgagactagacagatcacgaagcacgtggcgcagattctcgattcacgcatgaacacgaagtacgatgagaatgacaagctgatccgggaagtgaaggtcatcaccttgaagtcaaagctcgtttctgacttcaggaaggatttccaattttataaggtgcgcgagatcaacaattatcaccatgctcatgacgcatacctcaacgctgtggtcggaacagcattgattaagaagtacccgaagctcgagtccgaattcgtgtacggtgactataaggtttacgatgtgcgcaagatgatcgccaagtcagagcaggaaattggcaaggccactgcgaagtatttcttttactctaacattatgaatttctttaagactgagatcacgctggctaatggcgaaatccggaagagaccacttattgagaccaacggcgagacaggggaaatcgtgtgggacaaggggagggatttcgccacagtccgcaaggttctctctatgcctcaagtgaatattgtcaagaagactgaagtccagacgggcgggttctcaaaggaatctattctgcccaagcggaactcggataagcttatcgccagaaagaaggactgggacccgaagaagtatggaggtttcgactcaccaacggtggcttactctgtcctggttgtggcaaaggtggagaagggaaagtcaaagaagctcaagtctgtcaaggagctcctgggtatcaccattatggagaggtccagcttcgaaaagaatccgatcgattttctcgaggcgaagggatataaggaagtgaagaaggacctgatcattaagcttccaaagtacagtcttttcgagttggaaaacggcaggaagcgcatgttggcttccgcaggagagctccagaagggtaacgagcttgctttgccgtccaagtatgtgaacttcctctatctggcatcccactacgagaagctcaagggcagcccagaggataacgaacagaagcaactgtttgtggagcaacacaagcattatcttgacgagatcattgaacagatttcggagttcagtaagcgcgtcatcctcgccgacgcgaatttggataaggttctctcagcctacaacaagcaccgggacaagcctatcagagagcaggcggaaaatatcattcatctcttcaccctgacaaaccttggggctcccgctgcattcaagtattttgacactacgattgatcggaagagatacacttctacgaaggaggtgctggatgcaacccttatccaccaatcgattactggcctctacgagacgcggatcgacttgagtcagctcgggggggataagagaccagcggcaaccaagaaggcaggacaagcgaagaagaagaagtag

SEQ ID NO: 15 - DNA, 367 bp, Cauliflower mosaic virus
cggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggtt

SEQ ID NO: 16 - DNA, 742 bp, Oryza sativa
ctcattagcggtatgcatgttggtagaagtcggagatgtaaataattttcattatataaaaaaggtacttcgagaaaaataaatgcatacgaattaattctttttatgttttttaaaccaagtatatagaatttattgatggttaaaatttcaaaaatatgacgagagaaaggttaaacgtacggcatatacttctgaacagagagggaatatggggtttttgttgctcccaacaattcttaagcacgtaaaggaaaaaagcacattatccacattgtacttccagagatatgtacagcattacgtaggtacgttttctttttcttcccggagagatgatacaataatcatgtaaacccagaatttaaaaaatattctttactataaaaattttaattagggaacgtattattttttacatgacaccttttgagaaagagggacttgtaatatgggacaaatgaacaatttctaagaaatgggcatatgactctcagtacaatggaccaaattccctccagtcggcccagcaatacaaagggaaagaaatgagggggcccacaggccacggcccacttttctccgtggtggggagatccagctagaggtccggcccacaagtggcccttgccccgtgggacggtgggattgcagagcgcgtgggcggaaacaacagtttagtaccacctcgctcacgcaacgacgcgaccacttgcttataagctgctgcgctgaggctcag

SEQ ID NO: 17 - DNA, 83 bp, Artificial Sequence
  Nucleotide sequence of gRNA scaffold
gttttagagctagaaatagcaagttaaaataaggctagtccgttatcaacttgaaaaagtggcaccgagtcggtgcttttttt

SEQ ID NO: 18 - DNA, 20 bp, Artificial Sequence
  Nucleotide sequence of gRNA1
tggcgaagcgacctctcact

SEQ ID NO: 19 - DNA, 20 bp, Artificial Sequence
  Nucleotide sequence of gRNA2
cttgcaagctggtgggatcg

SEQ ID NO: 20 - DNA, 20 bp, Artificial Sequence
  Nucleotide sequence of gRNA3
ccacctatgtcgatcctagt

SEQ ID NO: 21 - DNA, 20 bp, Artificial Sequence
  Nucleotide sequence of gRNA4
agcaaccacggtatatcgaa

SEQ ID NO: 22 - DNA, 20 bp, Artificial Sequence
  Nucleotide sequence of gRNA5
ccgatcccctggcacgcact

SEQ ID NO: 23 - DNA, 21 bp, Artificial Sequence
  Nucleotide sequence of gRNA6
ggtttcatgagttttactaaa

SEQ ID NO: 24 - DNA, 20 bp, Artificial Sequence
  Nucleotide sequence of gRNA7
gtggaggtgtgactatcggc

SEQ ID NO: 25 - DNA, 20 bp, Artificial Sequence
  Nucleotide sequence of gRNA8
cgtttcagcaatagcattat

SEQ ID NO: 26 - DNA, 20 bp, Artificial Sequence
  Nucleotide sequence of gRNA9
ttattgttaaatttcagcaa

SEQ ID NO: 27 - DNA, 20 bp, Artificial Sequence
  Nucleotide sequence of gRNA10
cggtccagtcaagaccaagc

SEQ ID NO: 28 - DNA, 20 bp, Artificial Sequence
  Nucleotide sequence of gRNA11
aaataatttttctgcgcgga

SEQ ID NO: 29 - DNA, 20 bp, Artificial Sequence
  Nucleotide sequence of gRNA12
caaggatcttctgcgctggc

SEQ ID NO: 30 - DNA, 20 bp, Artificial Sequence
  Nucleotide sequence of gRNA13
agcacaatctagatggagtt

SEQ ID NO: 31 - DNA, 20 bp, Artificial Sequence
  Nucleotide sequence of gRNA14
tgaggagtcgccaaattatc

SEQ ID NO: 32 - DNA, 20 bp, Artificial Sequence
  Nucleotide sequence of gRNA15
taagttagctatagcaggtg

SEQ ID NO: 33 - DNA, 20 bp, Artificial Sequence
  Nucleotide sequence of gRNA16
acagggcaagttggggatga

SEQ ID NO: 34 - DNA, 20 bp, Artificial Sequence
  Nucleotide sequence of gRNA17
ctcacgttcgacgtctccct

SEQ ID NO: 35 - DNA, 20 bp, Artificial Sequence
  Nucleotide sequence of gRNA18
ttaatggacccaggccccta

SEQ ID NO: 36 - DNA, 20 bp, Artificial Sequence
  Nucleotide sequence of gRNA19
tcgcgtgtgcttttcgagtt

SEQ ID NO: 37 - DNA, 20 bp, Artificial Sequence
  Nucleotide sequence of gRNA20
gattgccgtagaagttaaaa

SEQ ID NO: 38 - DNA, 20 bp, Artificial Sequence
  Nucleotide sequence of gRNA21
gccggccggatcaggattat

SEQ ID NO: 39 - DNA, 20 bp, Artificial Sequence
  Nucleotide sequence of gRNA22
ttgcgccaaccaccgtgttt

SEQ ID NO: 40 - DNA, 90 bp, Oryza sativa
cgatgatgatggattccccttcgtcttcccgccgagtgcgtgccaggggatcggcgccccggcgccaccggtgcacgagttccagttctt

SEQ ID NO: 41 - DNA, 91 bp, Oryza sativa
cgatgatgatggattccccttcgtcttcccgccgagtcgcgtgccaggggatcggcgccccggcgccaccggtgcacgagttccagttctt

SEQ ID NO: 42 - DNA, 91 bp, Oryza sativa
cgatgatgatggattccccttcgtcttcccgccgagttgcgtgccaggggatcggcgccccggcgccaccggtgcacgagttccagttctt

SEQ ID NO: 43 - DNA, 91 bp, Oryza sativa
cgatgatgatggattccccttcgtcttcccgccgagtagcgtgccaggggatcggcgccccggcgccaccggtgcacgagttccagttctt

SEQ ID NO: 44 - DNA, 91 bp, Oryza sativa
cgatgatgatggattccccttcgtcttcccgccgagtggcgtgccaggggatcggcgccccggcgccaccggtgcacgagttccagttctt

SEQ ID NO: 45 - DNA, 86 bp, Oryza sativa
cgatgatgatggattccccttcgtcttcccgccgagtgccaggggatcggcgccccggcgccaccggtgcacgagttccagttctt

SEQ ID NO: 46 - DNA, 88 bp, Oryza sativa
cgatgatgatggattccccttcgtcttcccgccgagtgtgccaggggatcggcgccccggcgccaccggtgcacgagttccagttctt

SEQ ID NO: 47 - DNA, 79 bp, Oryza sativa
cgatgatgatggattccccttcgtcttcccgccaggggatcggcgccccggcgccaccggtgcacgagttccagttctt

SEQ ID NO: 48 - DNA, 85 bp, Oryza sativa
cgatgatgatggattccccttcgtcttcccgccgagtccaggggatcggcgccccggcgccaccggtgcacgagttccagttctt

SEQ ID NO: 49 - DNA, 54 bp, Oryza sativa
tgcgtgccaggggatcggcgccccggcgccaccggtgcacgagttccagttctt

SEQ ID NO: 50 - DNA, 90 bp, Oryza sativa; identical to SEQ ID NO: 40

SEQ ID NO: 51 - DNA, 90 bp, Oryza sativa
ccagaaaagcctatgccgagatgaggccccgagtgagaggtcgcttcgccaaagaacctgatcaggaagctgtcgcaccgccatccacct

SEQ ID NO: 52 - DNA, 91 bp, Oryza sativa; identical to SEQ ID NO: 42

SEQ ID NO: 53 - DNA, 91 bp, Oryza sativa
ccagaaaagcctatgccgagatgaggccccgagttgagaggtcgcttcgccaaagaacctgatcaggaagctgtcgcaccgccatccacct

SEQ ID NO: 54 - DNA, 91 bp, Oryza sativa; identical to SEQ ID NO: 43

SEQ ID NO: 55 - DNA, 91 bp, Oryza sativa; identical to SEQ ID NO: 53

SEQ ID NO: 56 - DNA, 91 bp, Oryza sativa; identical to SEQ ID NO: 43

SEQ ID NO: 57 - DNA, 91 bp, Oryza sativa
ccagaaaagcctatgccgagatgaggccccgagtagagaggtcgcttcgccaaagaacctgatcaggaagctgtcgcaccgccatccacct

SEQ ID NO: 58 - DNA, 91 bp, Oryza sativa; identical to SEQ ID NO: 42

SEQ ID NO: 59 - DNA, 91 bp, Oryza sativa; identical to SEQ ID NO: 57

SEQ ID NO: 60 - DNA, 90 bp, Oryza sativa; identical to SEQ ID NO: 40

SEQ ID NO: 61 - DNA, 91 bp, Oryza sativa; identical to SEQ ID NO: 53

SEQ ID NO: 62 - DNA, 81 bp, Oryza sativa
cgatgatgatggattccccttcgtcttcccgccgagtgcgtgccaggggatggcgccaccggtgcacgagttccagttctt

SEQ ID NO: 63 - DNA, 91 bp, Oryza sativa; identical to SEQ ID NO: 53

SEQ ID NO: 64 - DNA, 74 bp, Oryza sativa
cgatgatgatggattccccttcgtcttccgccaggggatcggcgcatgccaccggtgcacgagttccagttctt

SEQ ID NO: 65 - DNA, 91 bp, Oryza sativa; identical to SEQ ID NO: 53

SEQ ID NO: 66 - DNA, 48 bp, Oryza sativa
cgatgatgatggattcccggcgccaccggtgcacgagttccagttctt

SEQ ID NO: 67 - DNA, 91 bp, Oryza sativa; identical to SEQ ID NO: 53

SEQ ID NO: 68 - DNA, 104 bp, Oryza sativa
cgatgatgatggattccccttcgtcttcccgccgagtgtcaggggatcggcgcgtgccaggggatcggcgccccggcgccaccggtgcacgagttccagttctt

SEQ ID NO: 69 - DNA, 91 bp, Oryza sativa; identical to SEQ ID NO: 53